(19) United States
(12) Patent Application Publication
(10) Pub. No.: US 2016/0158261 A1
(43) Pub. Date: Jun. 9, 2016

(54) Antibiotic Kit and Composition and Uses Thereof

(71) Applicant: Foamix Pharmaceuticals Ltd., Rehovot (IL)

(72) Inventors: Doron Friedman, Karmei Yosef (IL); Alex Besonov, Rehovot (IL); Dov Tamarkin, Maccabim (IL); Meir Eini, Ness Ziona (IL)

(21) Appl. No.: 14/955,298

(22) Filed: Dec. 1, 2015

Related U.S. Application Data
(63) Continuation of application No. 11/448,490, filed on Jun. 7, 2006, now Pat. No. 9,211,259.
Application No. 11/448,490 is a continuation-in-part of application No. 10/532,618, filed on Dec. 22, 2005.
Application No. 10/532,618 is a continuation of application No. 11/448,490, filed on Jun. 7, 2006, now Pat. No. 9,211,259.
Application No. 11/448,490 is a continuation-in-part of application No. 10/911,367, filed on Aug. 4, 2004, now abandoned.
(60) Provisional application No. 60/429,546, filed on Nov. 29, 2002.
(60) Provisional application No. 60/492,385, filed on Aug. 4, 2003.
(60) Provisional application No. 60/688,244, filed on Jun. 7, 2005.

(30) Foreign Application Priority Data
Oct. 25, 2002 (IL) 152486

(51) Int. Cl. A61K 31/70; A61K 9/12; A61K 31/4164; A61K 31/4412; A61K 31/20; A61K 31/155; A61K 9/107

(52) U.S. Cl. CPC A61K 31/70; A61K 31/155; A61K 9/122; A61K 9/107; A61K 31/4412; A61K 31/20; A61K 31/4164

(57) ABSTRACT

The present invention relates to a therapeutic kit to provide a safe and effective dosage of an antibiotic agent, including an aerosol packaging assembly including: a container accommodating a pressurized product; and an outlet capable of releasing the pressurized product as a foam, wherein the pressurized product comprises a foamable composition including: an antibiotic agent; at least one organic carrier selected from the group consisting of a hydrophobic organic carrier, an organic polar solvent, an emollient and mixtures thereof, at a concentration of about 2% to about 50% by weight, a surface-active agent, about 0.01% to about 5% by weight of at least one polymeric additive selected from the group consisting of a bioadhesive agent, a gelling agent, a film forming agent and a phase change agent, water; and liquefied or compressed gas propellant at a concentration of about 3% to about 25% by weight of the total composition.

### CROSS REFERENCE TO RELATED APPLICATIONS

This application is a continuation-in-part application of co-pending U.S. patent application Ser. No. 10/532,618, filed Apr. 25, 2005, which is a national stage application of International Patent Application No. IB03/005527, designating the United States and filed on Oct. 24, 2003, which claims the benefit of priority under 35 U.S.C. §119(e) to U.S. Patent Application Ser. No. 60/429,546, filed on Nov. 29, 2002, both entitled “Cosmetic and Pharmaceutical Foam,” and which claims the benefit of priority under 35 U.S.C. §119(a) to Israeli Patent Application No. 152486, filed Oct. 25, 2002, all of which are hereby incorporated in their entirety by reference.

This application is a continuation-in-part application of co-pending U.S. patent application Ser. No. 10/911,367, filed on Aug. 4, 2004, which claims the benefit of priority under 35 U.S.C. §119(e) to U.S. Patent Application Ser. No. 60/492,385, filed on Aug. 4, 2003, both entitled “Foam Carrier Containing Amphiphilic Copolymer Gelling Agent” and both hereby incorporated in their entirety by reference.

This application claims the benefit of priority under 35 U.S.C. §119(e) to U.S. Patent Application Ser. No. 60/688,244, filed on Jun. 7, 2005, entitled “Antibiotic Kit and Composition and Uses Thereof” and hereby incorporated in its entirety by reference.

### BACKGROUND OF THE INVENTION

Antibiotic agents have been used to relieve various systemic and superficial disorders. Classical treatment applications include skin infections, vaginal infections, and other disorders that involve a bacterial infection in their etiological factors.

Antibiotic agents are available in topical dosage form. Compositions containing antibiotic agents for topical treatment of dermatological disorders are available primarily in cream, lotion gel and ointment forms. Rubbing creams or ointments into the skin is inherently inefficient and difficult to achieve a constant and balanced application over large area of skin. Therefore, while semi-solid compositions, such as creams, lotions, gels and ointments are commonly used by consumers, new forms are desirable in order to achieve better control of the application, while maintaining or bestowing the skin beneficial properties of such products. Hence, the development of new compositions, having breakable foam consistency when released from a container and liquid properties when applied onto the skin is advantageous.

Foams and, in particular, foam emulsions are complicated systems which do not form under all circumstances. Changes in foam emulsion composition, such as by the addition of active ingredients, may destabilize the foam.

PCT/AU99/00735 teaches a pharmaceutical foam composition including (a) an active ingredient; (b) an occlusive agent; (c) an aqueous solvent; and (d) an organic cosolvent, in which the active ingredient is insoluble in water and insoluble in both water and the occlusive agent, and wherein there is sufficient occlusive agent to form an occlusive layer on the skin.

US Published Application No. 2004/0151671 provides pharmaceutical compositions in a pressurized container, comprising a quick breaking alcoholic foaming agent.

### SUMMARY OF THE INVENTION

The present invention relates to a therapeutic kit to provide a safe and effective dosage of an antibiotic agent, including an aerosol packaging assembly including:

a) a container accommodating a pressurized product; and

b) an outlet capable of releasing the pressurized product as a foam;

wherein the pressurized product comprises a foamable composition including:
i. an antibiotic agent;
ii. at least one organic carrier selected from the group consisting of a hydrophobic organic carrier, an organic polar solvent, an emollient and mixtures thereof, at a concentration of about 2% to about 50% by weight;
iii. a surface-active agent;
iv. about 0.01% to about 5% by weight of at least one polymeric additive selected from the group consisting of a bioadhesive agent, a gelling agent, a film forming agent and a phase change agent;
v. water; and
vi. liquefied or compressed gas propellant at a concentration of about 3% to about 25% by weight of the total composition.

In one or more embodiments, the composition is selected from the group consisting of an oil-in-water emulsion and a water-in-oil emulsion.

In one or more embodiments the kit contains a valve, which is optionally attached to metered dose device.

In one or more embodiments the kit further includes a therapeutically active foam adjuvant is selected from the group consisting of a fatty alcohol having 15 or more carbons in their carbon chain; a fatty acid having 16 or more carbons in their carbon chain; fatty alcohols, derived from beeswax and including a mixture of alcohols, a majority of which has at least 20 carbon atoms in their carbon chain; a fatty alcohol having at least one double bond; a fatty acid having at least one double bond; a branched fatty alcohol; a branched fatty acid and a fatty acid substituted with a hydroxyl group.

In one or more embodiments, the composition further contains a penetration enhancer.

The kit according to the present invention can optionally further contain at least one additional therapeutic agent selected from the group consisting of a steroidal anti-inflammatory agent, an immunosuppressive agent, an immunomodulator, an immunoregulating agent, a hormonal agent, an antifungal agent, an antiviral agent, an antiparasitic agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, benzoyl chloride, calcium hypochlorite, magnesium hypochlorite, an anti-wrinkle agent, a radical scavenger, a metal, silver, a metal oxide, titanium dioxide, zinc oxide, zirconium oxide, iron oxide, silicone oxide, talc, carbon, an anti wrinkle agent, a skin whitening agent, a skin protective agent, a masking agent, an anti-wart agent, a refatting agent, a lubricating agent and mixtures thereof.

In further embodiments, the present invention provides a method of treating, alleviating or preventing disorders of the skin, body cavity or mucosal surface, wherein the disorder involves inflammation as one of its etiological factors, including administering topically to a subject having the disorder, a foamed composition including:
(1) an antibiotic agent;
(2) at least one organic carrier selected from a hydrophobic organic carrier, a polar solvent, an emollient and mixtures thereof, at a concentration of about 2% to about 50% by weight;
(3) about 0.1% to about 5% by weight of a surface-active agent;
(4) about 0.01% to about 5% by weight of a polymeric additive selected from a bioadhesive agent, a gelling agent, a film forming agent and a phase change agent; and
(5) water,
   wherein the antibiotic agent is administered in a therapeutically effective amount.

In one or more embodiments, the disorder to be treated is selected from the group consisting of a dermatose, a dermatitis, a vaginal disorder, a vulvar disorder, an anal disorder, a disorder of a body cavity, an ear disorder, a disorder of the nose, a disorder of the respiratory system, a bacterial infection, fungal infection, viral infection, dermatosis, dermatitis, parasitic infections, disorders of hair follicles and sebaceous glands, scaling papular diseases, benign tumors, malignant tumors, reactions to sunlight, bullous diseases, pigmentation disorders, disorders of cornification, pressure sores, disorders of sweating, inflammatory reactions, xerosis, ichthyosis, allergy, burn, wound, cut, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, osteoarthritis, joint pain, hormonal disorder, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum;

### BRIEF DESCRIPTION OF THE DRAWING

The invention is described with reference to the FIGURE which is presented for the purpose of illustration and are not intended to be limiting of the invention.

FIG. 1 is a schematic illustration of an aerosol valve suitable for use in the aerosol packaging assembly according to in one or more embodiments of the invention.

### DETAILED DESCRIPTION OF THE INVENTION

The present invention provides a therapeutic kit including an antibiotic agent. The kit includes an aerosol packaging assembly having a container accommodating a pressurized product and an outlet capable of releasing the pressurized product as a foam.

### Aerosol Packaging Assembly

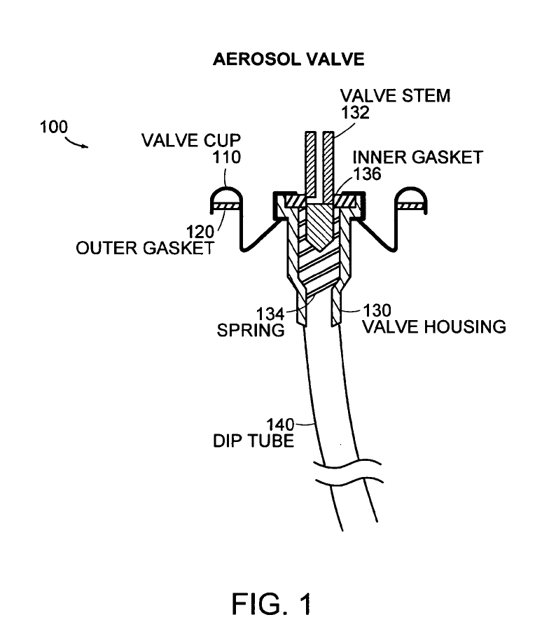

The aerosol packaging assembly typically includes a container suitable for accommodating a pressurized product and an outlet capable of releasing a foam. The outlet is typically a valve. FIG. 1 illustrates a typical aerosol valve 100. The valve is made up of the valve cup 110 typically constructed from tinplated steel, or aluminum, an outer gasket 120, which is the seal between the valve cup and the aerosol can (not shown), a valve housing 130, which contains the valve stem 132, spring 134 and inner gasket 136, and a dip tube 140, which allows the liquid to enter valve. The valve stem is the tap through which the product flows. The inner gasket 136 covers the aperture 150 (hole) in the valve stem. The valve spring 134 is usually made of stainless steel.

The valve stem is fitted with small apertures 150 (also termed “orifices” and “holes”), through which the product flows. Valves may contain one, two, three, four or more apertures, depending on the nature of the product to be dispensed. In the closed position, the aperture(s) is covered by the inner gasket. When the actuator is depressed it pushes the valve stem through the inner gasket, and the aperture(s) is uncovered, allowing liquid to pass through the valve and into the actuator.

The valve can have a stem with 1 to 4 apertures, or 1 to 2 apertures. Each aperture can have a diameter of about 0.2 mm to about 1 mm, or a diameter of about 0.3 mm to about 0.8 mm. The total aperture area, i.e., the sum of areas of all apertures in a given stem, is between about 0.01 mm² and 1 mm² or the total aperture area is between about 0.04 mm² and 0.5 mm².

In order to provide proper therapy, precise dosing is desired. According to one or more embodiments, the valve is attached, directly, or through a tube, to a metered dose device, which for dispensing an accurate dose of drug in the form of a foam. The metered dose valve is selected to release a foam in a volume that provides an adequate therapeutic dose to the target site of the skin, a body surface, a body cavity or mucosal surface, e.g., the mucosa of the nose, mouth, eye, ear, respiratory system, vagina or rectum.

In one or more embodiments, the meter dose valve provides a unit dose of between about 10 μL and about 1000 μL. Assuming a representative foam density (specific gravity) of 0.06 g/mL, a 10 μL valve provides a volume of about 0.17 mL of foam, and a 1000 μL metered dose valve provides about 17 mL of foam. Thus, by selecting a specific metered dosing valve and adjusting the foam density by fine tuning formulation parameters and adjusting the ratio between the liquid components of the composition and the propellant, one can design an adequate dosage form according to the specific target site. Exemplary metered dose devices may be found in co-pending application Ser. No. 11/406,133, entitled “Apparatus and Method for Releasing a Measured Amount of Content from a Container,” filed Apr. 18, 2006, which is hereby incorporated in its entirety by reference.

### Pharmaceutical Composition

All % values are provided on a weight (w/w) basis.

According to one or more embodiments of the present invention, the foamable therapeutic composition for administration to the skin, a body surface, a body cavity, a mucosal surface, the nose, the mouth, the eye, the ear canal, the respiratory system, the vagina and the rectum (severally and interchangeably termed herein “target site”) includes:

(1) an antibiotic agent, wherein the antibiotic agent is effective in the treatment of a disorder of the target site;

(2) at least one organic carrier selected from a hydrophobic organic carrier, a polar solvent, an emollient and mixtures thereof, at a concentration of about 2% to about 5%, or about 5% to about 10%; or about 10% to about 20%; or about 20% to about 50% by weight;

(3) about 0.1% to about 5% by weight of a surface-active agent;

(4) about 0.01% to about 5% by weight of at least one polymeric agent selected from a bioadhesive agent, a gelling agent, a film forming agent and a phase change agent; and

(5) a liquefied or compressed gas propellant at a concentration of about 3% to about 25% by weight of the total composition.

Water and optional ingredients are added to complete the total mass to 100%. Upon release from an aerosol container, the foamable composition forms an expanded foam suitable for topical administration.

According to one or more embodiments, the foamable composition is substantially alcohol-free, i.e., free of short chain alcohols. Short chain alcohols, having up to 5 carbon atoms in their carbon chain skeleton and one hydroxyl group, such as ethanol, propanol, isopropanol, butanol, iso-butanol, t-butanol and pentanol, are considered less desirable solvents or polar solvents due to their skin-irritating effect. This disadvantage is particularly meaningful in the case of an antibiotic treatment, which is often directed to open wounds and damaged skin and mucosal tissues. Thus, in one or more embodiments, the composition is substantially alcohol-free and includes less than about 5% final concentration of lower alcohols, preferably less than about 2%, more preferably less than about 1%.

In one or more embodiments, the foam composition is formulated as an oil-in-water emulsion or oil-in-water microemulsion, yet, in additional embodiments, the foam composition is formulated as an water-in-oil emulsion or water-in-oil microemulsion.

In one or more embodiments, the concentration of surface-active agent about 0.1% to about 5%, or from about 0.2% to about 2%.

In the context of the present invention, an antibiotic agent is a substance that has the capacity to inhibit the growth of or to destroy bacteria and other microorganisms.

In one or more embodiments, the antibiotic agent is selected from the classes consisting of beta-lactam antibiotics, aminoglycosides, ansa-type antibiotics, anthraquinones, antibiotic azoles, antibiotic glycopeptides, macrolides, antibiotic nucleosides, antibiotic peptides, antibiotic polyenes, antibiotic polyethers, quinolones, antibiotic steroids, sulfonamides, tetracycline, dicarboxylic acids, antibiotic metals, oxidizing agents, substances that release free radicals and/or active oxygen, cationic antimicrobial agents, quaternary ammonium compounds, biguanides, triguanides, bisbiguanides and analogs and polymers thereof and naturally occurring antibiotic compounds.

Beta-lactam antibiotics include, but are not limited to, 2-(3-alanyl)clavam, 2-hydroxymethylclavam, 8-epi-thienamycin, acetyl-thienamycin, amoxicillin, amoxicillin sodium, amoxicillin trihydrate, amoxicillin-potassium clavulanate combination, ampicillin, ampicillin sodium, ampicillin trihydrate, ampicillin-sulbactam, apalcillin, aspoxicillin, azidocillin, azlocillin, aztreonam, bacampicillin, biapenem, carbenicillin, carbenicillin disodium, carfecillin, carindacillin, carpetimycin, cefacetril, cefaclor, cefadroxil, cefalexin, cefaloridine, cefalotin, cefamandole, cefamandole, cefapirin, cefatrizine, cefatrizine propylene glycol, cefazedone, cefazolin, cefbuperazone, cefcapene, cefcapene pivoxil hydrochloride, cefdinir, cefditoren, cefditoren pivoxil, cefepime, cefetamet, cefetamet pivoxil, cefixime, cefmenoxime, cefmetazole, cefminox, cefminox, cefmolexin, cefodizime, cefonicid, cefoperazone, ceforanide, cefoselis, cefotaxime, cefotetan, cefotiam, cefoxitin, cefozopran, cefpiramide, cefpirome, cefpodoxime, cefpodoxime proxetil, cefprozil, cefquinome, cefradine, cefroxadine, cefsulodin, ceftazidime, cefteram, cefteram pivoxil, ceftezole, ceftibuten, ceftizoxime, ceftriaxone, cefuroxime, cefuroxime axetil, cephalosporin, cephamycin, chitinovorin, ciclacillin, clavulanic acid, clometocillin, cloxacillin, cycloserine, deoxy pluracidomycin, dicloxacillin, dihydro pluracidomycin, epicillin, epithienamycin, ertapenem, faropenem, flomoxef, flucloxacillin, hetacillin, imipenem, lenampicillin, loracarbef, mecillinam, meropenem, metampicillin, meticillin, mezlocillin, moxalactam, nafcillin, northienamycin, oxacillin, panipenem, penamecillin, penicillin, phenethicillin, piperacillin, tazobactam, pivampicillin, pivcefalexin, pivmecillinam, pivmecillinam hydrochloride, pluracidomycin, propicillin, sarmoxicillin, sulbactam, sulbenicillin, talampicillin, temocillin, terconazole, thienamycin, ticarcillin and analogs, salts and derivatives thereof.

Aminoglycosides include, but are not limited to, 1,2′-N-DL-isoseryl-3′,4′-dideoxykanamycin B, 1,2′-N-DL-isoseryl-kanamycin B, 1,2′-N—[(S)-4-amino-2-hydroxybutyryl]-3′,4′-dideoxykanamycin B, 1,2′-N—[(S)-4-amino-2-hydroxybutyryl]-kanamycin B, 1-N-(2-Aminobutanesulfonyl) kanamycin A, 1-N-(2-aminoethanesulfonyl)3′,4′-dideoxyribostamycin, 1-N-(2-Aminoethanesulfonyl)3′-deoxyribostamycin, 1-N-(2-aminoethanesulfonyl)3′4′-dideoxykanamycin B, 1-N-(2-aminoethanesulfonyl)kanamycin A, 1-N-(2-aminoethanesulfonyl)kanamycin B, 1-N-(2-aminoethanesulfonyl)ribostamycin, 1-N-(2-aminopropanesulfonyl)3′-deoxykanamycin B, 1-N-(2-aminopropanesulfonyl)3′4′-dideoxykanamycin B, 1-N-(2-aminopropanesulfonyl)kanamycin A, 1-N-(2-aminopropanesulfonyl)kanamycin B, 1-N-(L-4-amino-2-hydroxy-butyryl)2,′3′-dideoxy-2′-fluorokanamycin A, 1-N-(L-4-amino-2-hydroxy-propionyl)2,′3′-dideoxy-2′-fluorokanamycin A, 1-N-DL-3′,4′-dideoxy-isoserylkanamycin B, 1-N-DL-isoserylkanamycin, 1-N-DL-isoserylkanamycin B, 1-N-[L-(−)-(alpha-hydroxy-gamma-aminobutyryl)]-XK-62-2, 2′,3′-dideoxy-2′-fluorokanamycin A, 2-hydroxygentamycin A3, 2-hydroxygentamycin B, 2-hydroxygentamycin B1, 2-hydroxygentamycin JI-20A, 2-hydroxygentamycin JI-20B, 3″-N-methyl-4″-C-methyl-3′,4′-dodeoxy kanamycin A, 3″-N-methyl-4″-C-methyl-3′,4′-dodeoxy kanamycin B, 3″-N-methyl-4″-C-methyl-3′,4′-dodeoxy-6′-methyl kanamycin B, 3′,4′-Dideoxy-3′-eno-ribostamycin, 3′,4′-dideoxyneamine, 3′,4′-dideoxyribostamycin, 3′-deoxy-6′-N-methyl-kanamycin B, 3′-deoxyneamine, 3′-deoxyribostamycin, 3′-oxysaccharocin, 3,3′-nepotrehalosadiamine, 3-demethoxy-2″-N-formimidoylistamycin B disulfate tetrahydrate, 3-demethoxyistamycin B, 3-O-demethyl-2-N-formimidoylistamycin B, 3-O-demethylistamycin B, 3-trehalosamine, 4″,6″-dideoxydibekacin, 4-N-glycyl-KA-6606VI, 5″-Amino-3′,4′,5″-trideoxy-butirosin A, 6″-deoxydibekacin, 6′-epifortimicin A, 6-deoxy-neomycin (structure 6-deoxy-neomycin B), 6-deoxy-neomycin B, 6-deoxy-neomycin C, 6-deoxy-paromomycin, acmimycin, AHB-3′,4′-dideoxyribostamycin, AHB-3′-deoxykanamycin B, AHB-3′-deoxyneamine, AHB-3′-deoxyribostamycin, AHB-4″-6″-dideoxydibekacin, AHB-6″-deoxydibekacin, AHB-dideoxyneamine, AHB-kanamycin B, AHB-methyl-3′-deoxykanamycin B, amikacin, amikacin sulfate, apramycin, arbekacin, astromicin, astromicin sulfate, bekanamycin, bluensomycin, boholmycin, butirosin, butirosin B, catenulin, coumamidine gamma1, coumamidine gamma2,D,L-1-N-(alpha-hydroxy-beta-aminopropionyl)-XK-62-2, dactimicin, de-O-methyl-4-N-glycyl-KA-6606VI, de-O-methyl-KA-6606I, de-O-methyl-KA-70381, destomycin A, destomycin B, di-N6′,O3-demethylistamycin A, dibekacin, dibekacin sulfate, dihydrostreptomycin, dihydrostreptomycin sulfate, epi-formamidoylglycidylfortimicin B, epihygromycin, formimidoyl-istamycin A, formimidoyl-istamycin B, fortimicin B, fortimicin C, fortimicin D, fortimicin KE, fortimicin KF, fortimicin KG, fortimicin KG1 (stereoisomer KG1/KG2), fortimicin KG2 (stereoisomer KG1/KG2), fortimicin KG3, framycetin, framycetin sulphate, gentamicin, gentamycin sulfate, globeomycin, hybrimycin A1, hybrimycin A2, hybrimycin B1, hybrimycin B2, hybrimycin C1, hybrimycin C2, hydroxystreptomycin, hygromycin, hygromycin B, isepamicin, isepamicin sulfate, istamycin, kanamycin, kanamycin sulphate, kasugamycin, lividomycin, marcomycin, micronomicin, micronomicin sulfate, mutamicin, myomycin, N-demethyl-7-O-demethylcelesticetin, demethylcelesticetin, methanesulfonic acid derivative of istamycin, nebramycin, nebramycin, neomycin, netilmicin, oligostatin, paromomycin, quintomycin, ribostamycin, saccharocin, seldomycin, sisomicin, sorbistin, spectinomycin, streptomycin, tobramycin, trehalosmaine, trestatin, validamycin, verdamycin, xylostasin, zygomycin and analogs, salts and derivatives thereof.

Ansa-type antibiotics include, but are not limited to, 21-hydroxy-25-demethyl-25-methylthioprotostreptovaricin, 3-methylthiorifamycin, ansamitocin, atropisostreptovaricin, awamycin, halomicin, maytansine, naphthomycin, rifabutin, rifamide, rifampicin, rifamycin, rifapentine, rifaximin, rubradirin, streptovaricin, tolypomycin and analogs, salts and derivatives thereof.

Antibiotic anthraquinones include, but are not limited to, auramycin, cinerubin, ditrisarubicin, ditrisarubicin C, figaroic acid fragilomycin, minomycin, rabelomycin, rudolfomycin, sulfurmycin and analogs, salts and derivatives thereof.

Antibiotic azoles include, but are not limited to, azanidazole, bifonazole, butoconazol, chlormidazole, chiormidazole hydrochloride, cloconazole, cloconazole monohydrochloride, clotrimazol, dimetridazole, econazole, econazole nitrate, enilconazole, fenticonazole, fenticonazole nitrate, fezatione, fluconazole, flutrimazole, isoconazole, isoconazole nitrate, itraconazole, ketoconazole, lanoconazole, metronidazole, metronidazole benzoate, miconazole, miconazole nitrate, neticonazole, nimorazole, niridazole, omoconazol, ornidazole, oxiconazole, oxiconazole nitrate, propenidazole, secnidazol, sertaconazole, sertaconazole nitrate, sulconazole, sulconazole nitrate, tinidazole, tioconazole, voriconazol and analogs, salts and derivatives thereof.

Antibiotic glycopeptides include, but are not limited to, acanthomycin, actaplanin, avoparcin, balhimycin, bleomycin B (copper bleomycin), chloroorienticin, chloropolysporin, demethylvancomycin, enduracidin, galacardin, guanidylfungin, hachimycin, demethylvancomycin, N-nonanoyl-teicoplanin, phleomycin, platomycin, ristocetin, staphylocidin, talisomycin, teicoplanin, vancomycin, victomycin, xylocandin, zorbamycin and analogs, salts and derivatives thereof.

Macrolides include, but are not limited to, acetylleucomycin, acetylkitasamycin, angolamycin, azithromycin, bafilomycin, brefeldin, carbomycin, chalcomycin, cirramycin, clarithromycin, concanamycin, deisovaleryl-niddamycin, demycinosyl-mycinamycin, Di-O-methyltiacumicidin, dirithromycin, erythromycin, erythromycin estolate, erythromycin ethyl succinate, erythromycin lactobionate, erythromycin stearate, flurithromycin, focusin, foromacidin, haterumalide, haterumalide, josamycin, josamycin ropionate, juvenimycin, juvenimycin, kitasamycin, ketotiacumicin, lankavacidin, lankavamycin, leucomycin, machecin, maridomycin, megalomicin, methylleucomycin, methymycin, midecamycin, miocamycin, mycaminosyltylactone, mycinomycin, neutramycin, niddamycin, nonactin, oleandomycin, phenylacetyldeltamycin, pamamycin, picromycin, rokitamycin, rosaramicin, roxithromycin, sedecamycin, shincomycin, spiramycin, swalpamycin, tacrolimus, telithromycin, tiacumicin, tilmicosin, treponemycin, troleandomycin, tylosin, venturicidin and analogs, salts and derivatives thereof.

Antibiotic nucleosides include, but are not limited to, amicetin, angustmycin, azathymidine, blasticidin S, epiroprim, flucytosine, gougerotin, mildiomycin, nikkomycin, nucleocidin, oxanosine, oxanosine, puromycin, pyrazomycin, showdomycin, sinefungin, sparsogenin, spicamycin, tunicamycin, uracil polyoxin, vengicide and analogs, salts and derivatives thereof.

Antibiotic peptides include, but are not limited to, actinomycin, aculeacin, alazopeptin, amfomycin, amythiamycin, antifungal from Zalerion arboricola, antrimycin, apid, apidaecin, aspartocin, auromomycin, bacileucin, bacillomycin, bacillopeptin, bacitracin, bagacidin, beminamycin, beta-alanyl-L-tyrosine, bottromycin, capreomycin, caspofungine, cepacidine, cerexin, cilofungin, circulin, colistin, cyclodepsipeptide, cytophagin, dactinomycin, daptomycin, decapeptide, desoxymulundocandin, echanomycin, echinocandin B, echinomycin, ecomycin, enniatin, etamycin, fabatin, ferrimycin, ferrimycin, ficellomycin, fluoronocathiacin, fusaricidin, gardimycin, gatavalin, globopeptin, glyphomycin, gramicidin, herbicolin, iomycin, iturin, iyomycin, izupeptin, janiemycin, janthinocin, jolipeptin, katanosin, killertoxin, lipopeptide antibiotic, lipopeptide from Zalerion sp., lysobactin, lysozyme, macromomycin, magainin, melittin, mersacidin, mikamycin, mureidomycin, mycoplanecin, mycosubtilin, neopeptifluorin, neoviridogrisein, netropsin, nisin, nocathiacin, nocathiacin 6-deoxyglycoside, nosiheptide, octapeptin, pacidamycin, pentadecapeptide, peptifluorin, permetin, phytoactin, phytostreptin, planothiocin, plusbacin, polcillin, polymyxin antibiotic complex, polymyxin B, polymyxin B1, polymyxin F, preneocarzinostatin, quinomycin, quinupristin-dalfopristin, safracin, salmycin, salmycin, salmycin, sandramycin, saramycetin, siomycin, sperabillin, sporamycin, a streptomyces compound, subtilin, teicoplanin aglycone, telomycin, thermothiocin, thiopeptin, thiostrepton, tridecaptin, tsushimycin, tuberactinomycin, tuberactinomycin, tyrothricin, valinomycin, viomycin, virginiamycin, zervacin and analogs, salts and derivatives thereof.

In one or more embodiments, the antibiotic peptide is a naturally-occurring peptide that possesses an antibacterial and/or an antifungal activity. Such peptide can be obtained from a herbal or a vertebrate source.

Polyenes include, but are not limited to, amphotericin, amphotericin, aureofungin, ayfactin, azalomycin, blasticidin, candicidin, candicidin methyl ester, candimycin, candimycin methyl ester, chinopricin, filipin, flavofungin, fradicin, hamycin, hydropricin, levorin, lucensomycin, lucknomycin, mediocidin, mediocidin methyl ester, mepartricin, methylamphotericin, natamycin, niphimycin, nystatin, nystatin methyl ester, oxypricin, partricin, pentamycin, perimycin, pimaricin, primycin, proticin, rimocidin, sistomycosin, sorangicin, trichomycin and analogs, salts and derivatives thereof.

Polyethers include, but are not limited to, 20-deoxy-epi-narasin, 20-deoxysalinomycin, carriomycin, dianemycin, dihydrolonomycin, etheromycin, ionomycin, iso-lasalocid, lasalocid, lenoremycin, lonomycin, lysocellin, monensin, narasin, oxolonomycin, a polycyclic ether antibiotic, salinomycin and analogs, salts and derivatives thereof.

Quinolones include, but are not limited to, an alkyl-methylendioxy-4(1H)-oxocinnoline-3-carboxylic acid, alatrofloxacin, cinoxacin, ciprofloxacin, ciprofloxacin hydrochloride, danofloxacin, dermofongin A, enoxacin, enrofloxacin, fleroxacin, flumequine, gatifloxacin, gemifloxacin, grepafloxacin, levofloxacin, lomefloxacin, lomefloxacin, hydrochloride, miloxacin, moxifloxacin, nadifloxacin, nalidixic acid, nifuroquine, norfloxacin, ofloxacin, orbifloxacin, oxolinic acid, pazufloxacine, pefloxacin, pefloxacin mesylate, pipemidic acid, piromidic acid, premafloxacin, rosoxacin, rufloxacin, sparfloxacin, temafloxacin, tosufloxacin, trovafloxacin and analogs, salts and derivatives thereof.

Antibiotic steroids include, but are not limited to, aminosterol, ascosteroside, cladosporide A, dihydrofusidic acid, dehydro-dihydrofusidic acid, dehydrofusidic acid, fusidic acid, squalamine and analogs, salts and derivatives thereof.

Sulfonamides include, but are not limited to, chloramine, dapsone, mafenide, phthalylsulfathiazole, succinylsulfathiazole, sulfabenzamide, sulfacetamide, sulfachlorpyridazine, sulfadiazine, sulfadiazine silver, sulfadicramide, sulfadimethoxine, sulfadoxine, sulfaguanidine, sulfalene, sulfamazone, sulfamerazine, sulfamethazine, sulfamethizole, sulfamethoxazole, sulfamethoxypyridazine, sulfamonomethoxine, sulfamoxol, sulfanilamide, sulfaperine, sulfaphenazol, sulfapyridine, sulfaquinoxaline, sulfasuccinamide, sulfathiazole, sulfathiourea, sulfatolamide, sulfatriazin, sulfisomidine, sulfisoxazole, sulfisoxazole acetyl, sulfacarbamide and analogs, salts and derivatives thereof.

Tetracyclines include, but are not limited to, dihydrosteffimycin, demethyltetracycline, aclacinomycin, akrobomycin, baumycin, bromotetracycline, cetocyclin, chlortetracycline, clomocycline, daunorubicin, demeclocycline, doxorubicin, doxorubicin hydrochloride, doxycycline, lymecyclin, marcellomycin, meclocycline, meclocycline sulfosalicylate, methacycline, minocycline, minocycline hydrochloride, musettamycin, oxytetracycline, rhodirubin, rolitetracycline, rubomycin, serirubicin, steffimycin, tetracycline and analogs, salts and derivatives thereof.

Dicarboxylic acids, having between about 6 and about 14 carbon atoms in their carbon atom skeleton are particularly useful in the treatment of disorders of the skin and mucosal membranes that involve microbial. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid. Thus, in one or more embodiments of the present invention, dicarboxylic acids, having between about 6 and about 14 carbon atoms in their carbon atom skeleton, as well as their salts and derivatives (e.g., esters, amides, mercapto-derivatives, anhydraides), are useful immunomodulators in the treatment of disorders of the skin and mucosal membranes that involve inflammation. Azelaic acid and its salts and derivatives are preferred. It has antibacterial effects on both aerobic and anaerobic organisms, particularly propionibacterium acnes and staphylococcus epidermidis, normalizes keratinization, and has a cytotoxic effect on malignant or hyperactive melanocytes. In a preferred embodiment, the dicarboxylic acid is azelaic acid in a concentration greater than 10%. Preferably, the concentration of azelaic acid is between about 10% and about 25%. In such concentrates, azelaic acid is suitable for the treatment of a variety of skin disorders, such as acne, rosacea and hyperpigmentation.

In one or more embodiments, the antibiotic agent is an antibiotic metal. A number of metals ions been shown to possess antibiotic activity, including silver, copper, zinc, mercury, tin, lead, bismutin, cadmium, chromium and ions thereof. It has been theorized that these antibiotic metal ions exert their effects by disrupting respiration and electron transport systems upon absorption into bacterial or fungal cells. Anti-microbial metal ions of silver, copper, zinc, and gold, in particular, are considered safe for in vivo use. Anti-microbial silver and silver ions are particularly useful due to the fact that they are not substantially absorbed into the body.

Thus, in one or more embodiment, the antibiotic metal consists of an elemental metal, selected from the group consisting of silver, copper, zinc, mercury, tin, lead, bismutin, cadmium, chromium and gold, which is suspended in the composition as particles, microparticles, nanoparticles or colloidal particles. The antibiotic metal can further be intercalated in a chelating substrate.

In further embodiments, the antibiotic metal is ionic. The ionic antibiotic metal can be presented as an inorganic or organic salt (coupled with a counterion), an organometallic complex or an intercalate. Non binding examples of counter inorganic and organic ions are sulfadiazine, acetate, benzoate, carbonate, iodate, iodide, lactate, laurate, nitrate, oxide, palmitate, a negatively charged protein. In preferred embodiments, the antibiotic metal salt is a silver salt, such as silver acetate, silver benzoate, silver carbonate, silver iodate, silver iodide, silver lactate, silver laurate, silver nitrate, silver oxide, silver palmitate, silver protein, and silver sulfadiazine.

In one or more embodiments, the antibiotic metal or metal ion is embedded into a substrate, such as a polymer, a mineral (such as zeolite, clay and silica).

Oxidizing agents and substances that release free radicals and/or active oxygen. In one or more embodiments, the antibiotic agent comprises strong oxidants and free radical liberating compounds, such as oxygen, hydrogen peroxide, benzoyl peroxide, elemental halogen species, as well as oxygenated halogen species, bleaching agents (e.g., sodium, calcium or magnesium hypochloride and the like), perchlorite species, iodine, iodate, and benzoyl peroxide. Organic oxidizing agents are also included in the definition of “oxidizing agent” according to the present invention, such as quinones. Such agents possess a potent broad-spectrum activity

In one or more embodiments the antibiotic agent is a cationic antimicrobial agent. The outermost surface of bacterial cells universally carries a net negative charge, making them sensitive to cationic substances. Examples of cationic antibiotic agents include: quaternary ammonium compounds (QAC's)—QAC's are surfactants, generally containing one quaternary nitrogen associated with at least one major hydrophobic moiety; alkyltrimethyl ammonium bromides are mixtures of where the alkyl group is between 8 and 18 carbons long, such as cetrimide (tetradecyltrimethylammonium bromide); benzalkonium chloride, which is a mixture of n-alkyldimethylbenzyl ammonium chloride where the alkyl groups (the hydrophobic moiety) can be of variable length; dialkylmethyl ammonium halides; dialkylbenzyl ammonium halides; and QAC dimmers, which bear bi-polar positive charges in conjunction with interstitial hydrophobic regions.

In one or more embodiments, the antibiotic agent is selected from the group of biguanides, triguanides, bisbiguanides and analogs thereof.

Guanides, biguanides, biguanidines and triguanides are unsaturated nitrogen containing molecules that readily obtain one or more positive charges, which make them effective antimicrobial agents. The basic structures a guanide, a biguanide, a biguanidine and a triguanide are provided below.

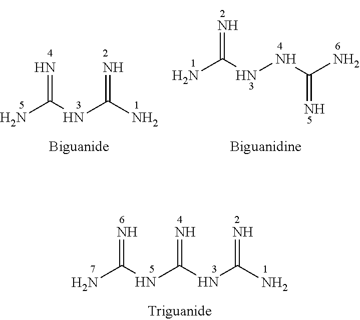 [SMILES: N=C(N)NC(=N)N ; N=C(N)NNC(=N)N ; N=C(N)NC(=N)NC(=N)N]

In one or more preferred embodiments, the guanide, biguanide, biguanidine or triguanide, provide bi-polar configurations of cationic and hydrophobic domains within a single molecule.

Examples of guanides, biguanides, biguanidines and triguanides that are currently been used as antibacterial agents include chlorhexidine and chlorohexidine salts, analogs and derivatives, such as chlorhexidine acetate, chlorhexidine gluconate and chlorhexidine hydrochloride, picloxydine, alexidine and polihexanide. Other examples of guanides, biguanides, biguanidines and triguanides that can conceivably be used according to the present invention are chlorproguanil hydrochloride, proguanil hydrochloride (currently used as antimalarial agents), metformin hydrochloride, phenformin and buformin hydrochloride (currently used as antidiabetic agents).

In one or more embodiments, the cationic antimicrobial agent is a polymer.

Cationic antimicrobial polymers include, for example, guanide polymers, biguanide polymers, or polymers having side chains containing biguanide moieties or other cationic functional groups, such as benzalkonium groups or quarternium groups (e.g., quaternary amine groups). It is understood that the term “polymer” as used herein includes any organic material comprising three or more repeating units, and includes oligomers, polymers, copolymers, block copolymers, terpolymers, etc. The polymer backbone may be, for example a polyethylene, polypropylene or polysilane polymer.

In one or more embodiments, the cationic antimicrobial polymer is a polymeric biguanide compound. When applied to a substrate, such a polymer is known to form a barrier film that can engage and disrupt a microorganism. An exemplary polymeric biguanide compound is polyhexamethylene biguanide (PHMB) salts. Other exemplary biguanide polymers include, but are not limited to poly(hexamethylenebiguanide), poly(hexamethylenebiguanide) hydrochloride, poly(hexamethylenebiguanide) gluconate, poly(hexamethylenebiguanide) stearate, or a derivative thereof. In one or more embodiments, the antimicrobial material is substantially water-insoluble.

Yet, in one or more embodiment, the antibiotic is a non-classified antibiotic agent, including, without limitation, aabomycin, acetomycin, acetoxycycloheximide, acetylnanaomycin, an actinoplanes sp. Compound, actinopyrone, aflastatin, albacarcin, albacarcin, albofungin, albofungin, alisamycin, alpha-R,S-methoxycarbonylbenzylmonate, altromycin, amicetin, amycin, amycin demanoyl compound, amycine, amycomycin, anandimycin, anisomycin, anthramycin, anti-syphilis imune substance, anti-tuberculosis imune substance, antibiotic from Eschericia coli, antibiotics from Streptomyces refuineus, anticapsin, antimycin, aplasmomycin, aranorosin, aranorosinol, arugomycin, ascofuranone, ascomycin, ascosin, Aspergillus flavus antibiotic, asukamycin, aurantinin, an Aureolic acid antibiotic substance, aurodox, avilamycin, azidamfenicol, azidimycin, bacillaene, a Bacillus larvae antibiotic, bactobolin, benanomycin, benzanthrin, benzylmonate, bicozamycin, bravomicin, brodimoprim, butalactin, calcimycin, calvatic acid, candiplanecin, carumonam, carzinophilin, celesticetin, cepacin, cerulenin, cervinomycin, chartreusin, chloramphenicol, chloramphenicol palmitate, chloramphenicol succinate sodium, chlorflavonin, chlorobiocin, chlorocarcin, chromomycin, ciclopirox, ciclopirox olamine, citreamicin, cladosporin, clazamycin, clecarmycin, clindamycin, coliformin, collinomycin, copiamycin, corallopyronin, corynecandin, coumermycin, culpin, cuprimyxin, cyclamidomycin, cycloheximide, dactylomycin, danomycin, danubomycin, delaminomycin, demethoxyrapamycin, demethylscytophycin, dermadin, desdamethine, dexylosyl-benanomycin, pseudoaglycone, dihydromocimycin, dihydronancimycin, diumycin, dnacin, dorrigocin, dynemycin, dynemycin triacetate, ecteinascidin, efrotomycin, endomycin, ensanchomycin, equisetin, ericamycin, esperamicin, ethylmonate, everninomicin, feldamycin, flambamycin, flavensomycin, florfenicol, fluvomycin, fosfomycin, fosfonochlorin, fredericamycin, frenolicin, fumagillin, fumifungin, funginon, fusacandin, fusafungin, gelbecidine, glidobactin, grahamimycin, granaticin, griseofulvin, griseoviridin, grisonomycin, hayumicin, hayumicin, hazymicin, hedamycin, heneicomycin, heptelicid acid, holomycin, humidin, isohematinic acid, karnatakin, kazusamycin, kristenin, L-dihydrophenylalanine, a L-isoleucyl-L-2-amino-4-(4′-amino-2′,5′-cyclohexadienyl) derivative, lanomycin, leinamycin, leptomycin, libanomycin, lincomycin, lomofungin, lysolipin, magnesidin, manumycin, melanomycin, methoxycarbonylmethylmonate, methoxycarbonylethylmonate, methoxycarbonylphenylmonate, methyl pseudomonate, methylmonate, microcin, mitomalcin, mocimycin, moenomycin, monoacetyl cladosporin, monomethyl cladosporin, mupirocin, mupirocin calcium, mycobacidin, myriocin, myxopyronin, pseudoaglycone, nanaomycin, nancimycin, nargenicin, neocarcinostatin, neoenactin, neothramycin, nifurtoinol, nocardicin, nogalamycin, novobiocin, octylmonate, olivomycin, orthosomycin, oudemansin, oxirapentyn, oxoglaucine methiodide, pactacin, pactamycin, papulacandin, paulomycin, phaeoramularia fungicide, phenelfamycin, phenyl, cerulenin, phenylmonate, pholipomycin, pirlimycin, pleuromutilin, a polylactone derivative, polynitroxin, polyoxin, porfiromycin, pradimicin, prenomycin, prop-2-enylmonate, protomycin, pseudomonas antibiotic, pseudomonic acid, purpuromycin, pyrinodemin, pyrrolnitrin, pyrrolomycin, amino, chloro pentenedioic acid, rapamycin, rebeccamycin, resistomycin, reuterin, reveromycin, rhizocticin, roridin, rubiflavin, naphthyridinomycin, saframycin, saphenamycin, sarkomycin, sarkomycin, sclopularin, selenomycin, siccanin, spartanamicin, spectinomycin, spongistatin, stravidin, streptolydigin, streptomyces arenae antibiotic complex, streptonigrin, streptothricins, streptovitacin, streptozotocine, a strobilurin derivative, stubomycin, sulfamethoxazol-trimethoprim, sakamycin, tejeramycin, terpentecin, tetrocarcin, thermorubin, thermozymocidin, thiamphenicol, thioaurin, thiolutin, thiomarinol, thiomarinol, tirandamycin, tolytoxin, trichodermin, trienomycin, trimethoprim, trioxacarcin, tyrissamycin, umbrinomycin, unphenelfamycin, urauchimycin, usnic acid, uredolysin, variotin, vermisporin, verrucarin and analogs, salts and derivatives thereof.

In one or more embodiments, the antibiotic agent is a naturally occurring antibiotic compound. As used herein, the term “naturally-occurring antibiotic agent” includes all antibiotic that are obtained, derived or extracted from plant or vertebrate sources. Non-limiting examples of families of naturally-occurring antibiotic agents include phenol, resorcinol, antibiotic aminoglycosides, anamycin, quinines, anthraquinones, antibiotic glycopeptides, azoles, macrolides, avilamycin, agropyrene, cnicin, aucubin antibioticsaponin fractions, berberine (isoquinoline alkaloid), arctiopicrin (sesquiterpene lactone), lupulone, humulone (bitter acids), allicin, hyperforin, echinacoside, coniosetin, tetramic acid, imanine and novoimanine.

Ciclopirox and ciclopiroxolamine possess fungicidal, fungistatic and sporicidal activity. They are active against a broad spectrum of dermatophytes, yeasts, moulds and other fungi, such as trichophyton species, microsporum species, epidermophyton species and yeasts (candida albicans, candida glabrata, other candida species and cryptococcus neoformans). Some aspergillus species are sensitive to ciclopirox as are some penicillium. Likewise, ciclopirox is effective against many gram-positive and gram-negative bacteria (e.g., escherichia coli, proteus mirabilis, pseudomonas aeruginosa, staphylococcus and streptococcus species), as well as mycoplasma species, trichomonas vaginalis and actinomyces.

Plant oils and extracts which contain antibiotic agents are also useful. Non limiting examples of plants that contain agents include thyme, perilla, lavender, tea tree, terfezia claveryi, Micromonospora, putterlickia verrucosa, putterlickia pyracantha putterlickia retrospinosa, Maytenus ilicifolia, maytenus evonymoides, maytenus aquifolia, faenia interjecta, cordyceps sinensis, couchgrass, holy thistle, plantain, burdock, hops, echinacea, buchu, chaparral, myrrh, red clover and yellow dock, garlic and St. John's wort.

Mixtures of these antibiotic agents may also be employed according to the present invention.

Solubility of the antibiotic agent is an important factor in the development of a stable foamable composition according to the present invention.

For definition purposes, in the context of the present invention, the descriptive terminology for solubility according to the US Pharmacopoeia (USP 23, 1995, p. 10), the European Pharmacopoeia (EP, 5^{th} Edition (2004), page 7) and several other textbooks used in the art of pharmaceutical sciences (see for example, Martindale, The Extra Pharmacopoeia, 30^{th} Edition (1993), page xiv of the Preface; and Remington's Pharmaceutical Sciences, 18^{th} Edition (1990), page 208) is adapted:

| | Parts of Solvent Required |
|---|---|
| Descriptive Term | for 1 Part of Solute |
| Very soluble | Less than 1 |
| Freely soluble | From 1 to 10 |
| Soluble | From 10 to 30 |
| Sparingly soluble | From 30 to 100 |
| Slightly soluble | From 100 to 1,000 |
| Very slightly soluble | From 1,000 to 10,000 |
| Practically insoluble or Insoluble | 10,000 and over |

Thus, in one or more embodiments, the antibiotic agent is “soluble”, “freely soluble” or “very soluble” (as defined above) in the aqueous phase of the emulsion. In other embodiments, where the agent possesses hydrophobic characteristics, the antibiotic agent is “soluble”, “freely soluble” or “very soluble” in the oil phase of the emulsion. In other cases, the antibiotic agent is “very slightly soluble”, “slightly soluble” or “sparingly soluble” in either the water phase or oil phase of the emulsion.

In other embodiments, the antibiotic agent is insoluble i.e., “requires 10,000 parts or more of a solvent to be solubilized”, in either the water phase of the composition, or the oil phase of the composition, but not in both.

In yet other embodiments, the antibiotic agent is not fully dissolved in both the aqueous phase of the oil phase of the emulsion concurrently, and thus, it is suspended in the emulsion (i.e., at least a portion of the antibiotic agent portion remains in solid state in the final composition). In such a case, the polymeric agents that are listed herein serve as suspension-stabilizing agents to stabilize the composition.

In certain embodiments of the present invention, the composition and properties of the aqueous phase of the emulsion (e.g., pH, electrolyte concentration and chelating agents) and/or the composition of the oil phase of the emulsion are adjusted to attain a desirable solubility profile of the active agent.

Antibiotic agents are useful for the treatment of skin infections and infections of other target sites, such as the vagina and rectum. The pH of the composition is adjusted for optimal efficacy, according to the specific infection and in light of the specific target site. In certain embodiments, the pH of the composition is between 3.5 and 8.5, and more preferably between about 4.5 and about 7.0, which is preferable for skin therapy. Yet, in other exemplary embodiments, the pH of the composition is between about 3 and about 4.5, which is suitable for vaginal therapy. In certain embodiments, the pH of the composition can be lower than 3. Two exemplary antibiotic agents that are being used both in skin therapy and vaginal therapy are metronidazole and clindamycin. For both agents, the pH of the foamable composition is adjusted between about 4.5 and about 7.0 for skin treatment and about 3 and about 4.5 for vaginal treatment. The adjustment of the pH can performed, as needed by the addition of an acid, a base or a buffering agent.

The antibiotic agent is included in the composition of the present invention in a concentration that provides a desirable ratio between the efficacy and safety. Typically, antibiotic agents are included in the composition in a concentration between about 0.005% and about 12%. However, in some embodiments, the concentration of between about 0.005% and about 0.5%, in other embodiment between about 0.5% and about 2%, and in additional embodiments between about 2% and about 5% or between about 5% and about 12%.

In one or more embodiments, the antibiotic agent is encapsulated in particles, microparticles, nanoparticles, microcapsules, spheres, microsphres, nanocapsules, nanospheres, liposomes, niosomes, polymer matrix, nanocrystals or microsponges.

In one or more embodiments, the antibiotic agent is an antibiotic agent precursor present at a concentration between about 0.05% and about 12%.

In one or more embodiments, the antibiotic agent is a compound that is positively identified using a laboratory method, suitable of detecting an antibiotic agent.

In one or more embodiments, the antibiotic agent is a substance that is positively identified using a competitive nuclear retinoic acid receptor-binding assay.

Several disorders of the target site (such as the skin, a body surface, a body cavity, a mucosal surface, the nose, the mouth, the eye, the ear canal, the respiratory system, the vagina and the rectum), involve a combination of etiological factors, some of which are related to a microbiological infection (that can be affected by an antibiotic agent); and other etiological factors that require an additional therapeutic modality. For example, impetigo involves bacterial infection as well as inflammation, and therefore combined treatment with an antibiotic agent and an anti-inflammatory agent would be beneficial. Likewise, chronic ulcers involve poor blood supply and potential bacterial, fungal and viral infections, which warrants a beneficial effect of a combination of an antibiotic agent and a vasoactive agent.

Additional non-limiting examples of combinations of an antibiotic agent and an additional active agent are provided in the following table:

| Disorder | Exemplary Additional Active Agent |
|---|---|
| acne | At least one agent selected from the group consisting of a |
| | retinoid; a keratolytic acid, an alpha-hydroxy acid and |
| | derivatives thereof, a beta-hydroxy acid and derivatives |
| | thereof, a skin-drying agent, an anti-seborrhea agent, a |
| | corticosteroid and a non-steroidal anti-inflammatory agent.. |
| Rosacea | At least one agent selected from the group consisting of a |
| | retinoid; a keratolytic acid, an alpha-hydroxy acid, a beta- |
| | hydroxy acid and derivatives thereof. |
| Otitis | At least one agent selected from the group of an antifungal |
| | agent, a local anesthetic agent, a corticosteroid and a non- |
| | steroidal anti-inflammatory agent. |
| Psoriasis | At least one agent selected from the group consisting of a |
| | corticosteroid, coal tar, anthralin and a photodynamic |
| | therapy agent |

Hence, in many cases, the inclusion of an additional therapeutic agent in the foamable composition of the present invention, contributes to the clinical activity of the antibiotic agent. Thus, in one or more embodiments, the foamable composition further includes at least one additional therapeutic agent, in a therapeutically effective concentration.

In one or more embodiments, the at least one additional therapeutic agent is selected from the group consisting of a steroidal anti-inflammatory agent, a nonsteroidal anti-inflammatory drug, an immunosuppressive agent, an immunomodulator, an immunoregulating agent, a hormonal agent, an antifungal agent, an antiviral agent, an antiparasitic agent, a vasoactive agent, a vasoconstrictor, a vasodilator, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, an antibiotic agent, an antiproliferative agent, an anticancer agent, a photodynamic therapy agent, an anti-wrinkle agent, a radical scavenger, a metal oxide (e.g., titanium dioxide, zinc oxide, zirconium oxide, iron oxide), silicone oxide, an anti wrinkle agent, a skin whitening agent, a skin protective agent, a masking agent, an anti-wart agent, a refatting agent, a lubricating agent and mixtures thereof.

In certain cases, the disorder to be treated involves unaesthetic lesions that need to be masked. For example, rosacea involves papules and pustules, which can be treated with an antibiotic agent, as well as erythema, telangiectasia and redness, which partially respond to treatment with an antibiotic agent. Thus, in one or more embodiments, the additional active agent is a masking agent, i.e., a pigment. Non limiting examples of suitable pigments include brown, yellow or red iron oxide or hydroxides, chromium oxides or hydroxides, titanium oxides or hydroxides, zinc oxide, FD&C Blue No. 1 aluminum lake, FD&C Blue No. 2 aluminum lake and FD&C Yellow No. 6 aluminum lake.

The foamable composition of the present invention can be an emulsion, or microemulsion, including an aqueous phase and an organic carrier phase. The organic carrier is selected from a hydrophobic organic carrier (also termed herein “hydrophobic solvent”), an emollient, a polar solvent, and a mixture thereof. The identification of a “solvent”, as used herein, is not intended to characterize the solubilization capabilities of the solvent for any specific active agent or any other component of the foamable composition. Rather, such information is provided to aid in the identification of materials suitable for use as a carrier in the foamable compositions described herein.

A “hydrophobic organic carrier” as used herein refers to a material having solubility in distilled water at ambient temperature of less than about 1 gm per 100 mL, more preferable less than about 0.5 gm per 100 mL, and most preferably less than about 0.1 gm per 100 mL. It is liquid at ambient temperature. The identification of a hydrophobic organic carrier or “hydrophobic solvent”, as used herein, is not intended to characterize the solubilization capabilities of the solvent for any specific active agent or any other component of the foamable composition. Rather, such information is provided to aid in the identification of materials suitable for use as a hydrophobic carrier in the foamable compositions described herein.

In one or more embodiments, the hydrophobic organic carrier is an oil, such as mineral oil. Mineral oil (Chemical Abstracts Service Registry number 8012-95-1) is a mixture of aliphatic, naphthalenic, and aromatic liquid hydrocarbons that derive from petroleum. It is typically liquid; its viscosity is in the range of between about 35 CST and about 100 CST (at 40° C.), and its pour point (the lowest temperature at which an oil can be handled without excessive amounts of wax crystals forming so preventing flow) is below 0° C. The hydrophobic organic carrier does not include thick or semi-solid materials, such as white petrolatum, also termed “Vaseline”, which, in certain compositions is disadvantageous due to its waxy nature and semi-solid texture.

According to one or more embodiments, hydrophobic solvents are liquid oils originating from vegetable, marine or animal sources. Suitable liquid oil includes saturated, unsaturated or polyunsaturated oils. By way of example, the unsaturated oil may be olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils or mixtures thereof, in any proportion.

Suitable hydrophobic solvents also include polyunsaturated oils containing poly-unsaturated fatty acids. In one or more embodiments, the unsaturated fatty acids are selected from the group of omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). Such unsaturated fatty acids are known for their skin-conditioning effect, which contribute to the therapeutic benefit of the present foamable composition. Thus, the hydrophobic solvent can include at least 6% of an oil selected from omega-3 oil, omega-6 oil, and mixtures thereof. In the context of the present invention, oils that possess therapeutically beneficial properties are termed “therapeutically active oil”.

Another class of hydrophobic solvents is the essential oils, which are also considered therapeutically active oil, which contain active biologically occurring molecules and, upon topical application, exert a therapeutic effect, which is conceivably synergistic to the beneficial effect of the antibiotic agent in the composition.

Another class of therapeutically active oils includes liquid hydrophobic plant-derived oils, which are known to possess therapeutic benefits when applied topically.

Silicone oils also may be used and are desirable due to their known skin protective and occlusive properties. Suitable silicone oils include non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. These are chosen from cyclic or linear polydimethylsiloxanes containing from about 3 to about 9, preferably from about 4 to about 5, silicon atoms. Volatile silicones such as cyclomethicones can also be used. Silicone oils are also considered therapeutically active oil, due to their barrier retaining and protective properties.

In one or more embodiments, the hydrophobic carrier includes at least 2% by weight silicone oil or at least 5% by weight.

The solvent may be a mixture of two or more of the above hydrophobic solvents in any proportion.

A further class of solvents includes “emollients” that have a softening or soothing effect, especially when applied to body areas, such as the skin and mucosal surfaces. Emollients are not necessarily hydrophobic. Examples of suitable emollients include hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof.

An additional class of emollients, suitable according to the present invention consists of polypropylene glycol (PPG) alkyl ethers, such as PPG stearyl ethers and PPG Butyl Ether, which are polypropylene ethers of stearyl ether that function as skin-conditioning agent in pharmaceutical and cosmetic formulations. PPG alkyl ethers can be incorporated in the foamable composition of the present invention in a concentration between about 1% and about 20%. The sensory properties of foams containing PPG alkyl ethers are favorable, as revealed by consumer panel tests. Surprisingly, it has been discovered that foams comprising PPG alkyl ethers are non-flammable, as shown in a test according to European Standard prEN 14851, titled “Aerosol containers—Aerosol foam flammability test”, while foams containing other oils are inflammable.

According to one or more embodiments of the present invention, the hydrophobic organic carrier includes a mixture of a hydrophobic solvent and an emollient. According to one or more embodiments, the foamable composition is a mixture of mineral oil and an emollient in a ratio between 2:8 and 8:2 on a weight basis.

A “polar solvent” is an organic solvent, typically soluble in both water and oil. Examples of polar solvents include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, sulfoxides, such as dimethylsulfoxide (DMSO), dimethylformanide, methyl dodecyl sulfoxide, dimethylacetamide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the polar solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

The polymeric agent serves to stabilize the foam composition and to control drug residence in the target organ. Exemplary polymeric agents are classified below in a non-limiting manner. In certain cases, a given polymer can belong to more than one of the classes provided below.

In one or more embodiments, the composition of the present invention includes at least one gelling agent. A gelling agent controls the residence of a therapeutic composition in the target site of treatment by increasing the viscosity of the composition, thereby limiting the rate of its clearance from the site. Many gelling agents are known in the art to possess mucoadhesive properties.

The gelling agent can be a natural gelling agent, a synthetic gelling agent and an inorganic gelling agent. Exemplary gelling agents that can be used in accordance with one or more embodiments of the present invention include, for example, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, starch, chemically modified starches and the like, semi-synthetic polymeric materials such as cellulose ethers (e.g. hydroxyethyl cellulose, methyl cellulose, carboxymethyl cellulose, hydroxy propylmethyl cellulose), guar gum, hydroxypropyl guar gum, soluble starch, cationic celluloses, cationic guars, and the like, and synthetic polymeric materials, such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like. Mixtures of the above compounds are contemplated.

Further exemplary gelling agents include the acrylic acid/ethyl acrylate copolymers and the carboxyvinyl polymers sold, for example, by the B.F. Goodrich Company under the trademark of Carbopol® resins. These resins consist essentially of a colloidal water-soluble polyalkenyl polyether crosslinked polymer of acrylic acid crosslinked with from 0.75% to 2% of a crosslinking agent such as polyallyl sucrose or polyallyl pentaerythritol. Examples include Carbopol® 934, Carbopol® 940, Carbopol® 950, Carbopol® 980, Carbopol® 951 and Carbopol® 981. Carbopol® 934 is a water-soluble polymer of acrylic acid crosslinked with about 1% of a polyallyl ether of sucrose having an average of about 5.8 allyl groups for each sucrose molecule.

In one or more embodiment, the composition of the present invention includes at least one polymeric agent, which is a water-soluble cellulose ether. Preferably, the water-soluble cellulose ether is selected from the group consisting of methylcellulose, hydroxypropyl cellulose, hydroxypropyl methylcellulose (Methocel), hydroxyethyl cellulose, methylhydroxyethylcellulose, methylhydroxypropylcellulose, hydroxyethylcarboxymethylcellulose, carboxymethylcellulose and carboxymethylhydroxyethylcellulose. More preferably, the water-soluble cellulose ether is selected from the group consisting of methylcellulose, hydroxypropyl cellulose and hydroxypropyl methylcellulose (Methocel). In one or more embodiments, the composition includes a combination of a water-soluble cellulose ether; and a naturally occurring polymeric materials, selected from the group including xanthan gum, guar gum, carrageenan gum, locust bean gum and tragacanth gum.

Yet, in other embodiments, the gelling agent includes inorganic gelling agents, such as silicone dioxide (fumed silica).

Mucoadhesive/bioadhesion has been defined as the attachment of synthetic or biological macromolecules to a biological tissue. Mucoadhesive agents are a class of polymeric biomaterials that exhibit the basic characteristic of a hydrogel, i.e. swell by absorbing water and interacting by means of adhesion with the mucous that covers epithelia. Compositions of the present invention may contain a mucoadhesive macromolecule or polymer in an amount sufficient to confer bioadhesive properties. The bioadhesive macromolecule enhances the delivery of biologically active agents on or through the target surface. The mucoadhesive macromolecule may be selected from acidic synthetic polymers, preferably having at least one acidic group per four repeating or monomeric subunit moieties, such as poly(acrylic)- and/or poly(methacrylic) acid (e.g., Carbopol®, Carbomer®), poly(methylvinyl ether/maleic anhydride) copolymer, and their mixtures and copolymers; acidic synthetically modified natural polymers, such as carboxymethylcellulose (CMC); neutral synthetically modified natural polymers, such as (hydroxypropyl)methylcellulose; basic amine-bearing polymers such as chitosan; acidic polymers obtainable from natural sources, such as alginic acid, hyaluronic acid, pectin, gum tragacanth, and karaya gum; and neutral synthetic polymers, such as polyvinyl alcohol or their mixtures. An additional group of mucoadhesive polymers includes natural and chemically modified cyclodextrin, especially hydroxypropyl-β-cyclodextrin. Such polymers may be present as free acids, bases, or salts, usually in a final concentration of about 0.01% to about 0.5% by weight.

A suitable bioadhesive macromolecule is the family of acrylic acid polymers and copolymers, (e.g., Carbopol®). These polymers contain the general structure —[CH₂—CH(COOH)—]ₙ. Hyaluronic acid and other biologically-derived polymers may be used.

Exemplary bioadhesive or mucoadhesive macromolecules have a molecular weight of at least 50 kDa, or at least 300 kDa, or at least 1,000 kDa. Favored polymeric ionizable macromolecules have not less than 2 mole percent acidic groups (e.g., COOH, SO₃H) or basic groups (NH₂, NRH, NR₂), relative to the number of monomeric units. The acidic or basic groups can constitute at least 5 mole percent, or at least 10 mole percent, or at least 25, at least 50 more percent, or even up to 100 mole percent relative to the number of monomeric units of the macromolecule.

Yet, another group of mucoadhesive agent includes inorganic gelling agents such as silicon dioxide (fumed silica), including but not limited to, AEROSIL 200 (DEGUSSA).

Many mucoadhesive agents are known in the art to also possess gelling properties.

The foam composition may contain a film-forming component. The film-forming component may include at least one water-insoluble alkyl cellulose or hydroxyalkyl cellulose. Exemplary alkyl cellulose or hydroxyalkyl cellulose polymers include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross-linking agent may be used to modify the polymer's characteristics. For example, esters such as dibutyl or diethyl phthalate, amides such as diethyldiphenyl urea, vegetable oils, fatty acids and alcohols such as oleic and myristyl acid may be used in combination with the cellulose derivative.

In one or more embodiments, the composition of the present invention includes a phase change polymer, which alters the composition behavior from fluid-like prior to administration to solid-like upon contact with the target mucosal surface. Such phase change results from external stimuli, such as changes in temperature or pH and exposure to specific ions (e.g., Ca²⁺). Non-limiting examples of phase change polymers include poly(N-isopropylamide) and Poloxamer 407®.

The polymeric agent is present in an amount in the range of about 0.01% to about 5.0% by weight of the foam composition. In one or more embodiments, it is typically less than about 1 wt % of the foamable composition.

Surface-active agents (also termed “surfactants”) include any agent linking oil and water in the composition, in the form of emulsion. A surfactant's hydrophilic/lipophilic balance (HLB) describes the emulsifier's affinity toward water or oil. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average).

According to one or more embodiments of the present invention, the surface-active agent has a hydrophilic lipophilic balance (HLB) between about 9 and about 14, which is the required HLB (the HLB required to stabilize an O/W emulsion of a given oil) of most oils and hydrophobic solvents. Thus, in one or more embodiments, the composition contains a single surface active agent having an HLB value between about 9 and 14, and in one or more embodiments, the composition contains more than one surface active agent and the weighted average of their HLB values is between about 9 and about 14. Yet, in other embodiments, when a water in oil emulsion is desirable, the composition contains one or more surface active agents, having an HLB value between about 2 and about 9.

The surface-active agent is selected from anionic, cationic, nonionic, zwitterionic, amphoteric and ampholytic surfactants, as well as mixtures of these surfactants. Such surfactants are well known to those skilled in the therapeutic and cosmetic formulation art. Nonlimiting examples of possible surfactants include polysorbates, such as polyoxyethylene (20) sorbitan monostearate (Tween 60) and poly(oxyethylene) (20) sorbitan monooleate (Tween 80); poly(oxyethylene) (POE) fatty acid esters, such as Myrj 45, Myrj 49, Myrj 52 and Myrj 59; poly(oxyethylene)alkylyl ethers, such as poly(oxyethylene) cetyl ether, poly(oxyethylene) palmityl ether, polyethylene oxide hexadecyl ether, polyethylene glycol cetyl ether, brij 38, brij 52, brij 56 and brij W1; sucrose esters, partial esters of sorbitol and its anhydrides, such as sorbitan monolaurate and sorbitan monolaurate; mono or diglycerides, isoceteth-20, sodium methyl cocoyl taurate, sodium methyl oleoyl taurate, sodium lauryl sulfate, triethanolamine lauryl sulfate and betaines.

In one or more embodiments of the present invention, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. We have surprisingly found that non-ionic surfactants alone provide foams of excellent quality, i.e. a score of “E” according to the grading scale discussed herein below.

In one or more embodiments, the surface active agent includes a mixture of at least one non-ionic surfactant and at least one ionic surfactant in a ratio in the range of about 100:1 to 6:1. In one or more embodiments, the non-ionic to ionic surfactant ratio is greater than about 6:1, or greater than about 8:1; or greater than about 14:1, or greater than about 16:1, or greater than about 20:1.

In one or more embodiments of the present invention, a combination of a non-ionic surfactant and an ionic surfactant (such as sodium lauryl sulphate and cocamidopropylbetaine) is employed, at a ratio of between 1:1 and 20:1, or at a ratio of 4:1 to 10:1. The resultant foam has a low specific gravity, e.g., less than 0.1 g/ml.

In certain preferred embodiments, the surface active agent consistst of essentially a non-ionic surfactant or a combination of non-ionic surfactants.

It has been surprisingly discovered that the stability of the composition is especially pronounced when a combination of at least one non-ionic surfactant having HLB of less than 9 and at least one non-ionic surfactant having HLB of equal or more than 9 is employed. The ratio between the at least one non-ionic surfactant having HLB of less than 9 and the at least one non-ionic surfactant having HLB of equal or more than 9, is between 1:8 and 8:1, or at a ratio of 4:1 to 1:4. The resultant HLB of such a blend of at least two emulsifiers is between about 9 and about 14.

Thus, in an exemplary embodiment, a combination of at least one non-ionic surfactant having HLB of less than 9 and at least one non-ionic surfactant having HLB of equal or more than 9 is employed, at a ratio of between 1:8 and 8:1, or at a ratio of 4:1 to 1:4, wherein the HLB of the combination of emulsifiers is between about 9 and about 14.

In one or more embodiments of the present invention, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content, which have higher HLB values.

The total surface active agent is in the range of about 0.1 to about 5% of the foamable composition, and is typically less than about 2% or less than about 1%.

Preferably, a therapeutically effective foam adjuvant is included in the foamable compositions of the present invention to increase the foaming capacity of surfactants and/or to stabilize the foam. In one or more embodiments of the present invention, the foam adjuvant agent includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols, derived from beeswax and including a mixture of alcohols, a majority of which has at least 20 carbon atoms in their carbon chain, are especially well suited as foam adjuvant agents. The amount of the fatty alcohol required to support the foam system is inversely related to the length of its carbon chains. Foam adjuvants, as defined herein are also useful in facilitating improved spreadability and absorption of the composition.

In one or more embodiments of the present invention, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. As for fatty alcohols, the amount of fatty acids required to support the foam system is inversely related to the length of its carbon chain.

In one or more embodiments, a combination of a fatty acid and a fatty ester is employed.

Optionally, the carbon atom chain of the fatty alcohol or the fatty acid may have at least one double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

An important property of the fatty alcohols and fatty acids used in context of the composition of the present invention is related to their therapeutic properties per se. Long chain saturated and mono unsaturated fatty alcohols, e.g., stearyl alcohol, erucyl alcohol, arachidyl alcohol and behenyl alcohol (docosanol) have been reported to possess antiviral, antiinfective, antiproliferative and anti-inflammatory properties (see, U.S. Pat. No. 4,874,794). Longer chain fatty alcohols, e.g., tetracosanol, hexacosanol, heptacosanol, octacosanol, triacontanol, etc., are also known for their metabolism modifying properties and tissue energizing properties. Long chain fatty acids have also been reported to possess anti-infective characteristics.

Thus, in preferred embodiments of the present invention, a combined and enhanced therapeutic effect is attained by including both an antibiotic agent and a therapeutically effective foam adjuvant in the same composition, thus providing a simultaneous anti-inflammatory and antiinfective effect from both components. Furthermore, in a further preferred embodiment, the composition concurrently comprises an antibiotic agent, a therapeutically effective foam adjuvant and a therapeutically active oil, as detailed above. Such combination provides an even more enhanced therapeutic benefit. Thus, the foamable carrier, containing the foam adjuvant provides an extra therapeutic benefit in comparison with currently used vehicles, which are inert and non-active.

The foam adjuvant according to one or more preferred embodiments of the present invention includes a mixture of fatty alcohols, fatty acids and hydroxy fatty acids and derivatives thereof in any proportion, providing that the total amount is 0.1% to 5% (w/w) of the carrier mass. More preferably, the total amount is 0.4%-2.5% (w/w) of the carrier mass.

Optionally, the composition further contains a penetration enhancer. Non limiting examples of penetration enhancers include propylene glycol, butylene glycols, glycerol, pentaerythritol, sorbitol, mannitol, oligosaccharides, dimethyl isosorbide, monooleate of ethoxylated glycerides having about 8 to 10 ethylene oxide units, polyethylene glycol 200-600, transcutol, glycofurol and cyclodextrins.

The therapeutic foam of the present invention may further optionally include a variety of formulation excipients, which are added in order to fine-tune the consistency of the formulation, protect the formulation components from degradation and oxidation and modify their consistency. Such excipients may be selected, for example, from stabilizing agents, antioxidants, humectants, preservatives, colorant and odorant agents and other formulation components, used in the art of formulation.

Aerosol propellants are used to generate and administer the foamable composition as a foam. The total composition including propellant, foamable compositions and optional ingredients is referred to as the foamable carrier. The propellant makes up about 3% to about 25 wt % of the foamable carrier. Examples of suitable propellants include volatile hydrocarbons such as butane, propane, isobutane or mixtures thereof, and fluorocarbon gases.

Composition and Foam Physical Characteristics

A pharmaceutical or cosmetic composition manufactured using the foam carrier according to one or more embodiments of the present invention is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foam composition of the present invention creates a stable emulsion having an acceptable shelf life of at least one year, or at least two years at ambient temperature. A feature of a product for cosmetic or medical use is long-term stability. Propellants, which are a mixture of low molecular weight hydrocarbons, tend to impair the stability of emulsions. It has been observed, however, that emulsion foam compositions according to the present invention are surprisingly stable. Following accelerated stability studies, they demonstrate desirable texture; they form fine bubble structures that do not break immediately upon contact with a surface, spread easily on the treated area and absorb quickly.

The composition should also be free flowing, to allow it to flow through the aperture of the container, e.g., and aerosol container, and create an acceptable foam. Compositions containing semi-solid hydrophobic solvents, e.g., white petrolatum, as the main ingredients of the oil phase of the emulsion, exhibit high viscosity and poor flowability and are inappropriate candidates for a foamable composition.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administratable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

A further aspect of the foam is breakability. The breakable foam is thermally stable, yet breaks under sheer force. Sheer-force breakability of the foam is clearly advantageous over thermally induced breakability. Thermally sensitive foams immediately collapse upon exposure to skin temperature and, therefore, cannot be applied on the hand and afterwards delivered to the afflicted area.

Another property of the foam is specific gravity, as measured upon release from the aerosol can. Typically, foams have specific gravity of less than 0.1 g/mL or less than 0.05 g/mL.

Fields of Pharmaceutical Applications

The foamable composition of the present invention is suitable for administration to an inflicted area, in need of treatment, including, but not limited to the skin, a body surface, a body cavity, a mucosal surface, the nose, the mouth, the eye, the ear canal, the respiratory system, the vagina and the rectum (severally and interchangeably termed herein “target site”).

Antibiotic agents are initially thought to affect disorders that involve blood circulation abnormalities, yet, in many case, circulation lays a secondary, yet influential role, which must be taken into account in order to optimize treatment. For example, cutaneous malignant tumors are characterized by poor blood circulation, which make them less responsive to drug treatment, and therefore usage of an antibiotic agent would be beneficial to the cancer therapy.

Thus, by including an appropriate antibiotic agent and optionally, additional active agents in the composition, the kit and the composition of the present invention are useful in treating an animal or a patient having one of a variety of dermatological disorders (also termed “dermatoses”) and/or having any secondary condition resulting from infections, which disorders and/or conditions are classified in a non-limiting exemplary manner according to the following groups:

Any disorders that involve a microbiological infection, or disorders that respond to treatment with an antibiotic agent,

An infection, selected from the group of a bacterial infection, a fungal infection, a yeast infection, a viral infection and a parasitic infection.

Any one of a variety of dermatological disorders, including dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo.

Dermatitis including contact dermatitis, atopic dermatitis, seborrheic dermatitis, nummular dermatitis, chronic dermatitis of the hands and feet, generalized exfoliative dermatitis, stasis dermatitis; lichen simplex chronicus; diaper rash;

Bacterial infections including cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma;

Fungal Infections including dermatophyte infections, yeast Infections; parasitic Infections including scabies, pediculosis, creeping eruption;

Viral Infections, including, but not limited to herpes genitalis and herpes labialis;

Disorders of hair follicles and sebaceous glands including acne, rosacea, perioral dermatitis, hypertrichosis (hirsutism), alopecia, including male pattern baldness, alopecia areata, alopecia universalis and alopecia totalis; pseudofolliculitis barbae, keratinous cyst;

Scaling papular diseases including psoriasis, pityriasis rosea, lichen planus, pityriasis rubra pilaris;

Benign tumors including moles, dysplastic nevi, skin tags, lipomas, angiomas, pyogenic granuloma, seborrheic keratoses, dermatofibroma, keratoacanthoma, keloid;

Malignant tumors including basal cell carcinoma, squamous cell carcinoma, malignant melanoma, Paget's disease of the nipples, Kaposi's sarcoma;

Reactions to sunlight including sunburn, chronic effects of sunlight, photosensitivity;

Bullous diseases including pemphigus, bullous pemphigoid, dermatitis herpetiformis, linear immunoglobulin A disease;

Pigmentation disorders including hypopigmentation such as vitiligo, albinism and postinflammatory hypopigmentation and hyperpigmentation such as melasma (chloasma), drug-induced hyperpigmentation, postinflammatory hyperpigmentation;

Disorders of comification including ichthyosis, keratosis pilaris, calluses and corns, actinic keratosis;

Pressure sores;

Disorders of sweating; and

Inflammatory reactions including drug eruptions, toxic epidermal necrolysis; erythema multiforme, erythema nodosum, granuloma annulare.

According to one or more embodiments of the present invention, the compositions are also useful in the therapy of non-dermatological disorders by providing transdermal delivery of an active antibiotic agent that is effective against non-dermatological disorders.

The same advantage is expected when the composition is topically applied to a body cavity or mucosal surface (e.g., the mucosa of the nose and mouth, the eye, the ear canal, vagina or rectum) to treat conditions such aschlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

In an embodiment of the present invention, the composition is useful for the treatment of wound, ulcer and burn. This use is particularly important since the composition of the present invention preads easily on the afflicted area, without the need of extensive rubbing.

In light of the expansion of the foam upon administration, it is further suitable for the treatment and prevention of post-surgical adhesions. Adhesions are scars that form abnormal connections between tissue surfaces. Post-surgical adhesion formation is a natural consequence of surgery, resulting when tissue repairs itself following incision, cauterization, suturing, or other means of trauma. When comprising an antibiotic agent an optionally, appropriate protective agents, the foam is suitable for the treatment or prevention of post surgical adhesions.

The invention is described with reference to the following examples. This invention is not limited to these examples and experiments. Many variations will suggest themselves and are within the full-intended scope of the appended claims.

### EXAMPLES

### Example 1

### Foamable Oil in Water Emulsion Antibiotic Compositions

An emulsion base was prepared by incorporating Phase A and Phase B with thorough mixing at elevated temperature (60-80° C.), followed by homogenization and gradual cooling to RT.

| Ingredient | % w/w |
|---|---|
| Mineral oil | 6.00 |
| Isopropyl myristate | 6.00 |
| Glyceryl monostearate | 0.50 |
| Stearyl alcohol | 1.00 |
| Xanthan gum | 0.30 |
| Methocel K100M | 0.30 |
| Polysorbate 80 | 1.00 |
| PEG-40 stearate | 3.00 |
| Purified water | 81.10 |
| Preaservative | 0.80 |

The emulsion base was used to produce foamable antibiotic compositions as detailed below. The compositions were further examined for emulsion uniformity, emulsion stability, foam quality and density and found stable, and meeting the requirements of density between 0.01 and 0.1 g/mL and excellent (E) quality, as shown in the table below.

| | % w/w | % w/w | % w/w | % w/w |
|---|---|---|---|---|
| Emulsion Base | 91.00 | 91.00 | 91.00 | 82.00 |
| Chlorohexydin sol. 20% | 1.00 | — | — | 10.00 |
| Tea tree oil | — | 1.00 | — | — |
| Clindamicin HCL | — | — | 1.00 | — |
| Propellant | 8.00 | 8.00 | 8.00 | 8.00 |

Foam Properties

| | | | | |
|---|---|---|---|---|
| Emulsion uniformity | uniform. | uniform. | uniform. | uniform. |
| pH | 4.92 | 4.89 | 4.54 | 5.05 |
| Foam quality | E | E | E | E |
| Density | 0.0356 | 0.0417 | 0.0369 | 0.0328 |

### Example 2

### Foamable Oil in Water Emulsion Base Antibiotic Compositions Comprising Ciclopiroxolamine

The following compositions contain ciclopiroxolamine, as example of an antibiotic foams comprising different emulsion compositions.

| | CPO- | CPO- | CPO- |
|---|---|---|---|
| | 006 | 008 | 011 |
| Ingredient Name | % w/w | % w/w | % w/w |
| Ciclopiroxolamine | 1.00 | 1.00 | 1.00 |
| Glyceryl monostearate | 0.45 | 0.45 | 0.45 |
| Light mineral oil | 5.00 | 5.00 | — |
| Isopropyl myristate | 5.00 | 5.00 | — |
| Caprylic/capric triglyceride | — | — | 10.00 |
| Cyclomethicone | 2.00 | — | — |
| Dimethicone | — | — | — |
| Sorbitan stearate | 0.60 | 0.60 | 0.60 |
| Stearyl alcohol | 0.85 | 0.85 | 0.85 |
| Steareth-21 | 2.00 | — | 2.00 |
| Laureth-4 | — | — | — |
| Cocamide DEA | — | 0.50 | — |
| PEG-40 stearate | 2.60 | 2.60 | 2.60 |
| Xanthan gum | 0.26 | 0.26 | 0.26 |
| Methocel A15C | 0.30 | — | — |
| Methocel A4M | — | — | — |
| Methocel K100LV | — | 0.30 | 0.30 |
| Polysorbate 80 | 0.90 | 0.90 | 0.90 |
| Lactic acid | to pH | to pH | to pH |
| | 7.0 | 7.0 | 7.0 |
| Purified water | To 100 | To 100 | To 100 |

| | CPO- | CPO- | CPO- | CPO- |
|---|---|---|---|---|
| | 012 | 014 | 015 | 035 |
| Ingredient Name | % w/w | % w/w | % w/w | % w/w |
| Ciclopiroxolamine | 1.00 | 1.00 | 1.00 | 1.00 |
| Glyceryl monostearate | 0.45 | 0.45 | 0.45 | 0.45 |
| Octyldodecanol | — | 10.00 | 10.00 | — |
| Caprylic/capric triglyceride | 10.00 | — | — | 2.00 |
| Cyclomethicone | 2.00 | 2.00 | — | 5.00 |
| Sorbitan stearate | 0.60 | 0.60 | 0.60 | 1.00 |
| Stearyl alcohol | 0.85 | 0.85 | 0.85 | 1.20 |
| Steareth-21 | 2.00 | — | 2.00 | — |
| Laureth-4 | — | 2.00 | — | 2.60 |
| Cocamide DEA | — | — | — | — |
| PEG-40 stearate | 2.60 | 2.60 | 2.60 | 2.60 |
| Xanthan gum | 0.26 | 0.26 | 0.26 | 0.26 |
| Methocel A15C | — | — | — | — |
| Methocel A4M | — | — | 0.30 | 0.30 |
| Methocel K100LV | 0.30 | 0.30 | — | — |
| Polysorbate 80 | 0.90 | 0.90 | 0.90 | 0.90 |
| Lactic acid | to pH | to pH | to pH | to pH |
| | 7.0 | 7.0 | 7.0 | 7.0 |
| Purified water | To 100 | To 100 | To 100 | To 100 |

Notes:
The liquefied or gas propellant can be added at a concentration of about 3% to about 25%.
The compositions contain a variety of organic carriers.
In the majotrity of the compositions the surface active agents are solely non-ionic.
The same vehicles can be used as vehicle of several additional antibiotic agents, listed in the embodiments of the present invention

### Example 3

### Non-Flammable Foamable Oil in Water Emulsion Base Antibiotic Compositions, Containing PPG Alley Ether and Comprising Ciclopiroxolamine

| | CPO032 | CPO033 | CPO038 | CPO041 |
|---|---|---|---|---|
| Ingredient | % w/w | % w/w | % w/w | % w/w |
| Ciclopiroxolamine | 1.00 | 1.00 | 1.00 | 1.00 |
| PPG-15 stearyl ether | 15.00 | 15.00 | 15.00 | 15.00 |
| Isopropyl miristate | — | — | 3.00 | — |
| lanolin | — | 2.00 | — | — |
| Behenyl alcohol | 1.00 | 1.00 | 1.00 | 1.00 |
| Steareth-21 | 1.50 | — | — | — |
| Ceteareth 20 | — | 1.50 | 1.50 | 1.50 |
| Ceteth 2 | — | — | 2.00 | 2.00 |
| Laureth-4 | 2.00 | 2.00 | — | — |
| Carboximethyl cellulose | — | 0.50 | — | — |
| sodium | | | | |
| Carbomer 1342 | — | — | 0.05 | 0.05 |
| Methyl cellulose | 0.15 | — | 0.15 | 0.15 |
| Xanthan gum | 0.15 | — | — | — |
| Glycerin USP | 3.00 | 3.00 | 3.00 | 3.00 |
| Polyethylene glycol 400 | — | 5.00 | — | — |
| Propylene glycol | 5.00 | — | 5.00 | 5.00 |
| Sol. of NaOH (18%) | to pH 8.5 | to pH 8.5 | to pH 8.5 | to pH 8.5 |
| Purified water | To 100 | To 100 | To 100 | To 100 |

| | CPO-052 | CPO-056 | CPO-057 |
|---|---|---|---|
| Ingredient | % w/w | % w/w | % w/w |
| Ciclopiroxolamine | 1.00 | 1.00 | 1.00 |
| PPG-15 stearyl ether | 15.00 | 15.00 | 15.00 |
| Lanolin | — | — | 2.00 |
| Steareth-21 | — | 1.50 | — |
| Behenyl alcohol | 1.00 | 1.00 | 1.00 |
| Laureth-4 | — | 2.00 | 2.00 |
| Macrogolryl Cetostearyl Ether | — | — | 1.50 |
| Ceteareth 20 | 1.60 | — | — |
| Ceteth 2 | 2.00 | — | — |
| Pemulen TR2 | 0.05 | — | — |
| Methocel A4M | 0.16 | 0.15 | — |
| CMC Sodium | — | — | 0.50 |
| Xanthan Gum | — | 0.15 | — |
| Glycerin USP | 3.00 | 3.00 | 3.00 |
| Propylene glycol | 5.40 | 5.40 | 5.00 |
| Lactic acid | To pH 7.5 | To pH 7.5 | To pH 7.5 |
| Purified Water | To 100 | To 100 | To 100 |

Notes:
The liquefied or gas propellant can be added at a concentration of about 3% to about 25%.
The compositions contain a variety of organic carriers.
In the majority of the compositions the surface active agents are solely non-ionic.
The formulations contain glycerin and/or propylene glycol (polar solvents), which contribute to skin penetration of the antibiotic agent
The same vehicles can be used as vehicle of several additional antibiotic agents, listed in the embodiments of the present invention

### Example 4

### Inflammability Test

A test according to European Standard prEN 14851, titled “Aerosol containers—Aerosol foam flammability test” was performed on foam compositions CPO32, CPO33 and CPO41. Approximately 5 g of foam, mousse gel or paste is sprayed from the aerosol container on to a watch glass. An ignition source (a lighter) was placed at the base of the watch glass and any ignition and sustained combustion of the foam, mousse, gel or paste was observed. The test was carried out in a draught-free environment capable of ventilation, with the temperature controlled at 20±5° C. and relative humidity in the range of 30% to 80%. According to the standard, appearance of a stable flame which is at least 4 cm high and which is maintained for at least 2 seconds defines a product as “inflammable”.

Results: foam compositions CPO32, CPO33 and CPO41 were found “non-flammable”.

### Example 5

### Foamable Oil in Water Emulsion Base Antibiotic Compositions, Containing a Combination of Hexylene Glycol and Propylene Glycol and Comprising Ciclopiroxolamine

| | CPO-058 |
|---|---|
| Ingredient | % w/w |
| Ciclopiroxolamine | 1.00 |
| Hexylene glycol | 5.00 |
| Glyceryl Monostearate | 0.50 |
| Sorbitane Stearate | 0.65 |
| Stearyl Alcohol | 0.92 |
| Steareth-21 | 2.20 |
| PEG-40 Stearate | 2.85 |
| Behenyl alcohol | 1.00 |
| Methocel A4M | 0.35 |
| Xanthan Gum | 0.30 |
| Polysorbate 80 | 1.00 |
| Propylene glycol | 5.00 |
| Lactic acid | To pH 7.5 |
| Purified Water | To 100 |

Notes:
The liquefied or gas propellant can be added at a concentration of about 3% to about 25%.
The surface active agents are solely non-ionic.
The formulation contains hexylene glycol and propylene glycol (polar solvents), which contribute to skin penetration of the antibiotic agent
The same vehicle can be used as vehicle of several additional antibiotic agents, listed in the embosiments of the present invention

### Example 6

### Foamable Oil in Water Emulsion Base Antibiotic Compositions, Containing Azelaic Acid

| | AZL041 | AZL043 |
|---|---|---|
| Ingredient | % w/w | % w/w |
| Azelaic Acid | 15.00 | 15.00 |
| Caprylic/capric triglyceride | 10.00 | 10.00 |
| Cetostearyl alcohol | 1.00 | 1.00 |
| Glyceryl stearate | 0.50 | 0.50 |
| Cholesterol | 1.00 | — |
| Benzoic acid | 0.20 | 0.20 |
| Butylated hydroxytoluene | 0.10 | 0.10 |
| PEG-40 stearate | 2.60 | 2.60 |
| Methylcellulose (Methocel A4M) | 0.10 | 0.10 |
| Xanthan gum | 0.25 | 0.25 |
| Polysorbate 80 | 0.90 | 0.90 |
| PEG-400 | 5.00 | — |
| Dimethyl isosorbide | — | 5.00 |
| Propylene glycol | 5.00 | 10.00 |
| Sodium hydroxide (18% sol.) | To pH = 4.5 | To pH = 4.5 |
| Purified water | to 100 | to 100 |

Notes:
The liquefied or gas propellant can be added at a concentration of about 3% to about 25%.
The surface active agents are solely non-ionic.
The formulation contains PEG-400 or propylene glycol (polar solvents), which contribute to skin penetration of the antibiotic agent

### Example 7

### Foamable Oil in Water Emulsion Base Antibiotic Compositions, Containing Metronidazole

| | | MZ1 | MZ1 | MZ3 |
|---|---|---|---|---|
| | | % | % | % |
| Ingredient | Function | w/w | w/w | w/w |
| Metronidazole | | 0.75-2.0 | | |
| Mineral oil | Hydrophobic solvent | 6.0 | 30.0 | 30.0 |
| Isopropyl myristate | Hydrophobic solvent | 6.0 | | |
| PEG 40-Stearate | Emulsifier | 3.0 | 3.0 | 3.0 |
| Stearyl alcohol | Co-emulsifier; foam | 1.0 | 1.0 | 1.0 |
| | adjuvant | | | |
| Polysorbate 80 | Emulsifier | 1.0 | 1.0 | 1.0 |
| Glyceryl stearate | Co-emulsifier; foam | 0.5 | 0.5 | 0.5 |
| | adjuvant | | | |
| Cocamidopropylbetaine | Emulsifier | 0.5 | | 0.5 |
| Xanthan gum | Thickening agent; | 0.3 | 0.3 | 0.3 |
| | stabilizer | | | |
| Methylcellulose | Thickening agent; | 0.3 | 0.3 | 0.3 |
| | stabilizer | | | |
| Preservative | | As needed | | |
| Purified water | | To 100 | | |
| | | MZ4 | MZ5 | MZ6 |
| | | w/w | % | % |
| Ingredient | Function | % | w/w | w/w |
| Metronidazole | | 0.75-2.0 | | |
| Mineral oil | Hydrophobic solvent | 6.0 | 6.0 | 6.0 |
| Isopropyl myristate | Hydrophobic solvent | 6.0 | 6.0 | 6.0 |
| PEG 40-Stearate | Emulsifier | 3.0 | 3.0 | 3.0 |
| Stearyl alcohol | Co-emulsifier; foam | 1.0 | 1.0 | 1.0 |
| | adjuvant | | | |
| Polysorbate 80 | Emulsifier | 1.0 | 1.0 | 1.0 |
| Glyceryl stearate | Co-emulsifier; foam | 0.5 | 0.5 | 0.5 |
| | adjuvant | | | |
| Cocamidopropylbetaine | Emulsifier | 0.5 | | |
| Xanthan gum | Thickening agent; | 0.3 | 0.3 | 0.3 |
| | stabilizer | | | |
| Methylcellulose | Thickening agent; | 0.3 | 0.3 | 0.3 |
| | stabilizer | | | |
| Urea | | 2.0- | | |
| | | 20.0 | | |
| Lactic acid/ammonium | | | 2.0- | |
| lactate | | | 12.0 | |
| Hexylene glycol | | | | 2.0- |
| | | | | 10.0 |

| | |
|---|---|
| Preservative | As needed |
| Purified water | To 100 |

| | | MZ7 | MZ8 |
|---|---|---|---|
| | | w/w | % |
| Ingredient | Function | % | w/w |
| Metronidazole | | 0.75-2.0 | |
| Mineral oil | Hydrophobic solvent | 6.0 | 6.0 |
| Isopropyl myristate | Hydrophobic solvent | 6.0 | 6.0 |
| PEG 40-Stearate | Emulsifier | 3.0 | 3.0 |
| Stearyl alcohol | Co-emulsifier; foam | 1.0 | 1.0 |
| | adjuvant | | |
| Polysorbate 80 | Emulsifier | 1.0 | 1.0 |
| Glyceryl stearate | Co-emulsifier; foam | 0.5 | 0.5 |
| | adjuvant | | |
| Cocamidopropylbetaine | Emulsifier | 0.5 | 0.5 |
| Acrylates/C10-30 Alkyl | Thickening agent; | 0.3 | 0.3 |
| Acrylate Cross-Polymer | stabilizer | | |
| Microcrystalline cellulose | | | 0.3 |
| Methylcellulose | Thickening agent; | 0.3 | 0.3 |
| | stabilizer | | |
| Preservative | | As | As |
| | | needed | needed |
| TEA | Neutralizer | 0.1 | 0.1 |
| Purified water | | To 100 | To 100 |

Notes:
The liquefied or gas propellant can be added at a concentration of about 3% to about 25%.
Formulations MZ4, MZ5 and MZ6 contain urea and lactic acid, which are keratolytic. PEG-400 or propylene glycol (polar solvents), which contribute to skin penetration of the antibiotic agent
Formulation MZ6 contains hexylene glycol, which contribute to skin penetration of the antibiotic agent
All formulations are of “Excellent” quality foam
The concentration of metronidazole in the composition may be altered in the range of about 0.75% and about 2%.
It was found that formulations, comprising up to 2% metronidazole produced stable foams with “Good” to “Excellent” quality.
It was further surprisingly found that metronidazole is substantially dissolved in the foamable composition of the present invention, up to a concentration of about 1.8%, while it is known that the soluble concentration of Metronidazole is 0.75% and higher concentrations of metrnidazole are expected to be in suspension

1.-91.

92. A composition comprising a foamable carrier and a liquefied or compressed gas propellant in a canister, the carrier comprising:
i. an antibiotic agent;
ii. an unsaturated fatty acid selected from the group consisting of an omega-3 fatty acid, an omega-6 fatty acid, and mixtures of both;
iii. a fatty alcohol;
iv. an emollient;
v. a surface-active agent; and
vi. water;
wherein upon release a shear-sensitive foam is produced.

93. The composition of claim 92, wherein the water is in an amount selected from the group consisting of about 80% to about 98%, about 75% to about 95%, about 60% to about 90%, and about 25% to about 75% by weight of the carrier.

94. The composition of claim 93, wherein the carrier is an emulsion selected from the group consisting of an oil-in-water emulsion, and a water-in-oil emulsion.

95. The composition of claim 93, wherein the propellant is at a concentration of about 3% to about 25% by weight of the composition.

96. The composition of claim 92, wherein the unsaturated fatty acid comprises a linoleic acid, a linolenic acid, gamma-linoleic acid (GLA), eicosapentaenoic acid (EPA), docosahexaenoic acid (DHA), or mixtures of any two or more thereof.

97. The composition of claim 96, wherein the unsaturated fatty acid comprises docosahexaenoic acid (DHA).

98. The composition of claim 93, further comprising about 2% to about 50% by weight of the carrier of a hydrophobic organic carrier.

99. The composition of claim 98, wherein the hydrophobic organic carrier is present in an amount selected from the group consisting of (i) about 2% to about 5%; (ii) about 5% to about 10%; (iii) about 10% to about 20%; and (iv) about 20% to about 50% by weight of the carrier.

100. The composition of claim 99, wherein the hydrophobic organic carrier is selected from a group consisting of an olive oil, a mineral oil, isopropyl myristate, a corn oil, a soybean oil, a canola oil, a cottonseed oil, a sesame oil, a sunflower oil, a borage seed oil, a syzigium aromaticum oil, a hempseed oil, a herring oil, a cod-liver oil, a salmon oil, a flaxseed oil, a wheat germ oil, an evening primrose oil, an omega-3 oil, an omega-6 oil, a rosehip oil, a tea tree oil, a basil, a camphor oil, a cardamom oil, a carrot oil, a citronella oil, a clary sage oil, a clove oil, a cypress oil, a frankincense oil, a ginger oil, a grapefruit oil, a hyssop oil, a jasmine oil, a lavender oil, a lemon oil, a mandarin oil, a marjoram oil, a myrrh oil, a neroli oil, a nutmeg oil, a petitgrain oil, a sage oil, a tangerine oil, a vanilla oil, a verbena oil, a plant-derived oil, an isostearic acid derivative, isopropyl palmitate, lanolin oil, diisopropyl dimerate, a maleated soybean oil, octyl palmitate, isopropyl isostearate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, hydrogenated coco-glycerides, isononyl isononanoate, isotridecyl isononanoate, myristal myristate, triisocetyl citrate, octyl dodecanol, a sucrose ester of fatty acids, octyl hydroxystearate a silicone oil, a polyalkyl siloxane, a polyaryl siloxane, a polyalkylaryl siloxane, a polyether siloxane copolymer, a polydimethylsiloxane (Dimethicones), a poly(dimethylsiloxane)-(diphenyl-siloxane) copolymer, and mixtures of any two or more thereof.

101. The composition of claim 93, further comprising about 0.01% to about 5% by weight of the carrier of at least one polymeric additive selected from the group consisting of a bioadhesive agent, a gelling agent, a film forming agent, a phase change agent, and mixtures of any two or more thereof.

102. The composition of claim 95, wherein the fatty alcohol is selected from the group consisting of fatty alcohols having 15 or more carbons in their carbon chain; fatty alcohols derived from beeswax and including a mixture of alcohols, a majority of which has at least 20 carbon atoms in their carbon chain; fatty alcohols having at least one double bond; branched fatty alcohols; cetyl alcohol; stearyl alcohol; arachidyl alcohol; behenyl alcohol; 1-triacontanol; and mixtures of any two or more thereof.

103. The composition of claim 92, further comprising a fatty acid selected from the group consisting of fatty acids having 16 or more carbons in their carbon chain; fatty acids having at least one double bond; branched fatty acids; fatty acids substituted with a hydroxyl group; hexadecanoic acid; stearic acid; arachidic acid; behenic acid; octacosanoic acid; 12-hydroxy stearic acid; and mixtures of any two or more thereof.

104. The composition of claim 102, wherein the composition is substantially alcohol-free.

105. The composition of claim 102, wherein the antibiotic agent is selected from the group consisting of beta-lactam antibiotics, aminoglycosides, ansa-type antibiotics, anthraquinones, antibiotic azoles, antibiotic glycopeptides, macrolides, antibiotic nucleosides, antibiotic peptides, antibiotic polyenes, antibiotic polyethers, quinolones, antibiotic steroides, sulfonamides, tetracycline, dicarboxylic acids, antibiotic metals, oxidizing agents, substances that release free radicals or active oxygen, cationic antimicrobial agents, quaternary ammonium compounds, biguanides, triguanides, bisbiguanides and analogs and polymers thereof, and naturally occurring antibiotic compounds.

106. The composition of claim 105, wherein the antibiotic agent is selected from the group consisting of:
i. a beta-lactam, selected from the group consisting of 2-(3-alanyl)clavam, 2-hydroxymethylclavam, 8-epi-thienamycin, acetyl-thienamycin, amoxicillin, amoxicillin sodium, amoxicillin trihydrate, amoxicillin-potassium clavulanate combination, ampicillin, ampicillin sodium, ampicillin trihydrate, ampicillin-sulbactam, apalcillin, aspoxicillin, azidocillin, azlocillin, aztreonam, bacampicillin, biapenem, carbenicillin, carbenicillin disodium, carfecillin, carindacillin, carpetimycin, cefacetril, cefaclor, cefadroxil, cefalexin, cefaloridine, cefalotin, cefamandole, cefamandole, cefapirin, cefatrizine, cefatrizine propylene glycol, cefazedone, cefazolin, cefbuperazone, cefcapene, cefcapene pivoxil hydrochloride, cefdinir, cefditoren, cefditoren pivoxil, cefepime, cefetamet, cefetamet pivoxil, cefixime, cefmenoxime, cefmetazole, cefminox, cefminox, cefmolexin, cefodizime, cefonicid, cefoperazone, ceforanide, cefoselis, cefotaxime, cefotetan, cefotiam, cefoxitin, cefozopran, cefpiramide, cefpirome, cefpodoxime, cefpodoxime proxetil, cefprozil, cefquinome, cefradine, cefroxadine, cefsulodin, ceftazidime, cefteram, cefteram pivoxil, ceftezole, ceftibuten, ceftizoxime, ceftriaxone, cefuroxime, cefuroxime axetil, cephalosporin, cephamycin, chitinovorin, ciclacillin, clavulanic acid, clometocillin, cloxacillin, cycloserine, deoxy pluracidomycin, dicloxacillin, dihydro pluracidomycin, epicillin, epithienamycin, ertapenem, faropenem, flomoxef, flucloxacillin, hetacillin, imipenem, lenampicillin, loracarbef, mecillinam, meropenem, metampicillin, meticillin, mezlocillin, moxalactam, nafcillin, northienamycin, oxacillin, panipenem, penamecillin, penicillin, phenethicillin, piperacillin, tazobactam, pivampicillin, pivcefalexin, pivmecillinam, pivmecillinam hydrochloride, pluracidomycin, propicillin, sarmoxicillin, sulbactam, sulbenicillin, talampicillin, temocillin, terconazole, thienamycin, and ticarcillin;
ii. an aminoglycoside, selected from the group consisting of 1,2′-N-DL-isoseryl-3′,4′-dideoxykanamycin B, 1,2′-N-DL-isoseryl-kanamycin B, 1,2′-N—[(S)-4-amino-2-hydroxybutyryl]-3′,4′-dideoxykanamycin B, 1,2′-N—[(S)-4-amino-2-hydroxybutyryl]-kanamycin B, 1-N-(2-Aminobutanesulfonyl) kanamycin A, 1-N-(2-aminoethanesulfonyl)3′,4′-dideoxyribostamycin, 1-N-(2-Aminoethanesulfonyl)3′-deoxyribostamycin, 1-N-(2-aminoethanesulfonyl)3′4′-dideoxykanamycin B, 1-N-(2-aminoethanesulfonyl)kanamycin A, 1-N-(2-aminoethanesulfonyl)kanamycin B, 1-N-(2-aminoethanesulfonyl)ribostamycin, 1-N-(2-aminopropanesulfonyl)3′-deoxykanamycin B, 1-N-(2-aminopropanesulfonyl)3′4′-dideoxykanamycin B, 1-N-(2-aminopropanesulfonyl)kanamycin A, 1-N-(2-aminopropanesulfonyl)kanamycin B, 1-N-(L-4-amino-2-hydroxy-butyryl)2,′3′-dideoxy-2′-fluorokanamycin A, 1-N-(L-4-amino-2-hydroxy-propionyl)2,′3′-dideoxy-2′-fluorokanamycin A, 1-N-DL-3′,4′-dideoxy-isoserylkanamycin B, 1-N-DL-isoserylkanamycin, 1-N-DL-isoserylkanamycin B, 1-N-[L-(−)-(alpha-hydroxy-gamma-aminobutyryl)]-XK-62-2, 2′,3′-dideoxy-2′-fluorokanamycin A, 2-hydroxygentamycin A3, 2-hydroxygentamycin B, 2-hydroxygentamycin B1, 2-hydroxygentamycin JI-20A, 2-hydroxygentamycin JI-20B, 3″-N-methyl-4″-C-methyl-3′,4′-dodeoxy kanamycin A, 3″-N-methyl-4″-C-methyl-3′,4′-dodeoxy kanamycin B, 3″-N-methyl-4″-C-methyl-3′,4′-dodeoxy-6′-methyl kanamycin B, 3′,4′-Dideoxy-3′-eno-ribostamycin, 3′,4′-dideoxyneamine, 3′,4′-dideoxyribostamycin, 3′-deoxy-6′-N-methyl-kanamycin B, 3′-deoxyneamine, 3′-deoxyribostamycin, 3′-oxysaccharocin, 3,3′-nepotrehalosadiamine, 3-demethoxy-2″-N-formimidoylistamycin B disulfate tetrahydrate, 3-demethoxyistamycin B, 3-O-demethyl-2-N-formimidoylistamycin B, 3-O-demethylistamycin B, 3-trehalosamine, 4″,6″-dideoxydibekacin, 4-N-glycyl-KA-6606VI, 5″-Amino-3′,4′,5″-trideoxy-butirosin A, 6″-deoxydibekacin, 6′-epifortimicin A, 6-deoxy-neomycin (structure 6-deoxy-neomycin B), 6-deoxy-neomycin B, 6-deoxy-neomycin C, 6-deoxy-paromomycin, acmimycin, AHB-3′,4′-dideoxyribostamycin, AHB-3′-deoxykanamycin B, AHB-3′-deoxyneamine, AHB-3′-deoxyribostamycin, AHB-4″-6″-dideoxydibekacin, AHB-6″-deoxydibekacin, AHB-dideoxyneamine, AHB-kanamycin B, AHB-methyl-3′-deoxykanamycin B, amikacin, amikacin sulfate, apramycin, arbekacin, astromicin, astromicin sulfate, bekanamycin, bluensomycin, boholmycin, butirosin, butirosin B, catenulin, coumamidine gamma1, coumamidine gamma2,D,L-1-N-(alpha-hydroxy-beta-aminopropionyl)-XK-62-2, dactimicin, de-O-methyl-4-N-glycyl-KA-6606VI, de-O-methyl-KA-6606I, de-O-methyl-KA-7038I, destomycin A, destomycin B, di-N6′,O3-demethylistamycin A, dibekacin, dibekacin sulfate, dihydrostreptomycin, dihydrostreptomycin sulfate, epi-formamidoylglycidylfortimicin B, epihygromycin, formimidoyl-istamycin A, formimidoyl-istamycin B, fortimicin B, fortimicin C, fortimicin D, fortimicin KE, fortimicin KF, fortimicin KG, fortimicin KG1 (stereoisomer KG1/KG2), fortimicin KG2 (stereoisomer KG1/KG2), fortimicin KG3, framycetin, framycetin sulphate, gentamicin, gentamycin sulfate, globeomycin, hybrimycin A1, hybrimycin A2, hybrimycin B1, hybrimycin B2, hybrimycin C1, hybrimycin C2, hydroxystreptomycin, hygromycin, hygromycin B, isepamicin, isepamicin sulfate, istamycin, kanamycin, kanamycin sulphate, kasugamycin, lividomycin, marcomycin, micronomicin, micronomicin sulfate, mutamicin, myomycin, N-demethyl-7-O-demethylcelesticetin, demethylcelesticetin, methanesulfonic acid derivative of istamycin, nebramycin, nebramycin, neomycin, netilmicin, oligostatin, paromomycin, quintomycin, ribostamycin, saccharocin, seldomycin, sisomicin, sorbistin, spectinomycin, streptomycin, tobramycin, trehalosmaine, trestatin, validamycin, verdamycin, xylostasin, and zygomycin;
iii. an ansa-type antibiotics, selected from the group consisting of 21-hydroxy-25-demethyl-25-methylthioprotostreptovaricin, 3-methylthiorifamycin, ansamitocin, atropisostreptovaricin, awamycin, halomicin, maytansine, naphthomycin, rifabutin, rifamide, rifampicin, rifamycin, rifapentine, rifaximin, rubradirin, streptovaricin, and tolypomycin;
iv. an anthraquinone, selected from the group consisting of auramycin, cinerubin, ditrisarubicin, ditrisarubicin C, figaroic acid fragilomycin, minomycin, rabelomycin, rudolfomycin, and sulfurmycin;
v. an azole, selected from the group consisting of azanidazole, bifonazole, butoconazol, chlormidazole, chlormidazole hydrochloride, cloconazole, cloconazole monohydrochloride, clotrimazol, dimetridazole, econazole, econazole nitrate, enilconazole, fenticonazole, fenticonazole nitrate, fezatione, fluconazole, flutrimazole, isoconazole, isoconazole nitrate, itraconazole, ketoconazole, lanoconazole, metronidazole, metronidazole benzoate, miconazole, miconazole nitrate, neticonazole, nimorazole, niridazole, omoconazol, ornidazole, oxiconazole, oxiconazole nitrate, propenidazole, secnidazol, sertaconazole, sertaconazole nitrate, sulconazole, sulconazole nitrate, tinidazole, tioconazole, and voriconazol;
vi. a glycopeptide, selected from the group consisting of acanthomycin, actaplanin, avoparcin, balhimycin, bleomycin B (copper bleomycin), chloroorienticin, chloropolysporin, demethylvancomycin, enduracidin, galacardin, guanidylfungin, hachimycin, demethylvancomycin, N-nonanoyl-teicoplanin, phleomycin, platomycin, ristocetin, staphylocidin, talisomycin, teicoplanin, vancomycin, victomycin, xylocandin, and zorbamycin;
vii. a macrolide, selected from the group consisting of acetylleucomycin, acetylkitasamycin, angolamycin, azithromycin, bafilomycin, brefeldin, carbomycin, chalcomycin, cirramycin, clarithromycin, concanamycin, deisovaleryl-niddamycin, demycinosyl-mycinamycin, Di-O-methyltiacumicidin, dirithromycin, erythromycin, erythromycin estolate, erythromycin ethyl succinate, erythromycin lactobionate, erythromycin stearate, flurithromycin, focusin, foromacidin, haterumalide, haterumalide, josamycin, josamycin ropionate, juvenimycin, juvenimycin, kitasamycin, ketotiacumicin, lankavacidin, lankavamycin, leucomycin, machecin, maridomycin, megalomicin, methylleucomycin, methymycin, midecamycin, miocamycin, mycaminosyltylactone, mycinomycin, neutramycin, niddamycin, nonactin, oleandomycin, phenylacetyldeltamycin, pamamycin, picromycin, rokitamycin, rosaramicin, roxithromycin, sedecamycin, shincomycin, spiramycin, swalpamycin, tacrolimus, telithromycin, tiacumicin, tilmicosin, treponemycin, troleandomycin, tylosin, and venturicidin;
viii. a nucleoside, selected from the group consisting of amicetin, angustmycin, azathymidine, blasticidin S, epiroprim, flucytosine, gougerotin, mildiomycin, nikkomycin, nucleocidin, oxanosine, oxanosine, puromycin, pyrazomycin, showdomycin, sinefungin, sparsogenin, spicamycin, tunicamycin, uracil polyoxin, and vengicide;
ix. a peptide, selected from the group consisting of actinomycin, aculeacin, alazopeptin, amfomycin, amythiamycin, antifungal from Zalerion arboricola, antrimycin, apid, apidaecin, aspartocin, auromomycin, bacileucin, bacillomycin, bacillopeptin, bacitracin, bagacidin, berninamycin, beta-alanyl-L-tyrosine, bottromycin, capreomycin, caspofungine, cepacidine, cerexin, cilofungin, circulin, colistin, cyclodepsipeptide, cytophagin, dactinomycin, daptomycin, decapeptide, desoxymulundocandin, echanomycin, echinocandin B, echinomycin, ecomycin, enniatin, etamycin, fabatin, ferrimycin, ferrimycin, ficellomycin, fluoronocathiacin, fusaricidin, gardimycin, gatavalin, globopeptin, glyphomycin, gramicidin, herbicolin, iomycin, iturin, iyomycin, izupeptin, janiemycin, j anthinocin, j olipeptin, katanosin, killertoxin, lipopeptide antibiotic, lipopeptide from Zalerion sp., lysobactin, lysozyme, macromomycin, magainin, melittin, mersacidin, mikamycin, mureidomycin, mycoplanecin, mycosubtilin, neopeptifluorin, neoviridogrisein, netropsin, nisin, nocathiacin, nocathiacin 6-deoxyglycoside, nosiheptide, octapeptin, pacidamycin, pentadecapeptide, peptifluorin, permetin, phytoactin, phytostreptin, planothiocin, plusbacin, polcillin, polymyxin antibiotic complex, polymyxin B, polymyxin B1, polymyxin F, preneocarzinostatin, quinomycin, quinupristin-dalfopristin, safracin, salmycin, salmycin, salmycin, sandramycin, saramycetin, siomycin, sperabillin, sporamycin, a streptomyces compound, subtilin, teicoplanin aglycone, telomycin, thermothiocin, thiopeptin, thiostrepton, tridecaptin, tsushimycin, tuberactinomycin, tuberactinomycin, tyrothricin, valinomycin, viomycin, virginiamycin, and zervacin;
x. a naturally-occurring peptide that possesses at least an antibacterial, or an antifungal activity;
xi. a peptide obtained from a herbal, or a vertebrate source;
xii. a polyene, selected from the group consisting of amphotericin, amphotericin, aureofungin, ayfactin, azalomycin, blasticidin, candicidin, candicidin methyl ester, candimycin, candimycin methyl ester, chinopricin, filipin, flavofungin, fradicin, hamycin, hydropricin, levorin, lucensomycin, lucknomycin, mediocidin, mediocidin methyl ester, mepartricin, methylamphotericin, natamycin, niphimycin, nystatin, nystatin methyl ester, oxypricin, partricin, pentamycin, perimycin, pimaricin, primycin, proticin, rimocidin, sistomycosin, sorangicin, and trichomycin;
xiii. a polyether, selected from the group consisting of 20-deoxy-epi-narasin, 20-deoxysalinomycin, carriomycin, dianemycin, dihydrolonomycin, etheromycin, ionomycin, iso-lasalocid, lasalocid, lenoremycin, lonomycin, lysocellin, monensin, narasin, oxolonomycin, a polycyclic ether antibiotic, and salinomycin;
xiv. a quinolone, selected from the group consisting of alkyl-methylendioxy-4(1H)-oxocinnoline-3-carboxylic acid, alatrofloxacin, cinoxacin, ciprofloxacin, ciprofloxacin hydrochloride, danofloxacin, dermofongin A, enoxacin, enrofloxacin, fleroxacin, flumequine, gatifloxacin, gemifloxacin, grepafloxacin, levofloxacin, lomefloxacin, lomefloxacin, hydrochloride, miloxacin, moxifloxacin, nadifloxacin, nalidixic acid, nifuroquine, norfloxacin, ofloxacin, orbifloxacin, oxolinic acid, pazufloxacine, pefloxacin, pefloxacin mesylate, pipemidic acid, piromidic acid, premafloxacin, rosoxacin, rufloxacin, sparfloxacin, temafloxacin, tosufloxacin, trovafloxacin, and rovafloxacin;
xv. a steroid, selected from the group consisting of aminosterol, ascosteroside, cladosporide, dihydrofusidic acid, dehydro-dihydrofusidic acid, dehydrofusidic acid, fusidic acid, and squalamine;
xvi. a sulfonamide, selected from the group consisting of chloramine, dapsone, mafenide, phthalylsulfathiazole, succinylsulfathiazole, sulfabenzamide, sulfacetamide, sulfachlorpyridazine, sulfadiazine, sulfadiazine silver, sulfadicramide, sulfadimethoxine, sulfadoxine, sulfaguanidine, sulfalene, sulfamazone, sulfamerazine, sulfamethazine, sulfamethizole, sulfamethoxazole, sulfamethoxypyridazine, sulfamonomethoxine, sulfamoxol, sulfanilamide, sulfaperine, sulfaphenazol, sulfapyridine, sulfaquinoxaline, sulfasuccinamide, sulfathiazole, sulfathiourea, sulfatolamide, sulfatriazin, sulfisomidine, sulfisoxazole, sulfisoxazole acetyl, and sulfacarbamide;
xvii. a tetracycline, selected from the group consisting of dihydrosteffimycin, demethyltetracycline, aclacinomycin, akrobomycin, baumycin, bromotetracycline, cetocyclin, chlortetracycline, clomocycline, daunorubicin, demeclocycline, doxorubicin, doxorubicin hydrochloride, doxycycline, lymecyclin, marcellomycin, meclocycline, meclocycline sulfosalicylate, methacycline, minocycline, minocycline hydrochloride, musettamycin, oxytetracycline, rhodirubin, rolitetracycline, rubomycin, serirubicin, steffimycin, and tetracycline;
xviii. a dicarboxylic acid, selected from the group consisting of adipic acid, pimelic acid, suberic acid, azelaic acid, sebacic acid, 1,11-undecanedioic acid, 1,12-dodecanedioic acid, 1,13-tridecanedioic acid, and 1,14-tetradecanedioic acid.
xix. an antibiotic metal or a metal ion, wherein the metal is selected from the group consisting of silver, copper, zinc, mercury, tin, lead, bismutin, cadmium, chromium, and gold;
xx. a silver compound, selected from the group consisting of silver acetate, silver benzoate, silver carbonate, silver iodate, silver iodide, silver lactate, silver laurate, silver nitrate, silver oxide, silver palmitate, silver protein, and silver sulfadiazine;
xxi. an oxidizing agent or a substance that releases free radicals or active oxygen, wherein the oxidizing agent or the substance is selected from the group consisting of oxygen, hydrogen peroxide, benzoyl peroxide, elemental halogen species, as well as oxygenated halogen species, bleaching agents, sodium, calcium, magnesium hypochloride, perchlorite species, iodine, iodate, and benzoyl peroxide;
xxii. a cationic antimicrobial agent, selected from the group consisting of quaternary ammonium compounds, alkyltrimethyl ammonium bromide, cetrimide, benzalkonium chloride, n-alkyldimethylbenzyl ammonium chloride, dialkylmethyl ammonium halide, and dialkylbenzyl ammonium halide;
xxiii. a biguanide, a biguanidine or a triguanide having a skeleton selected from: 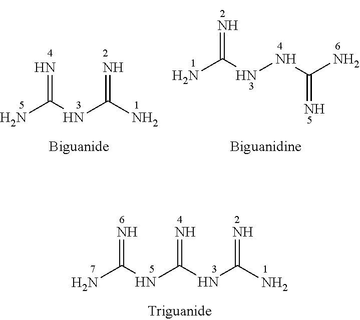 [SMILES: N=C(N)NC(=N)N ; N=C(N)NNC(=N)N ; N=C(N)NC(=N)NC(=N)N]
xxiv. a compound, selected from the group consisting of chlorhexidine acetate, chlorhexidine gluconate, chlorhexidine hydrochloride, picloxydine, alexidine, polihexanide, chlorproguanil hydrochloride, proguanil hydrochloride, metformin hydrochloride, phenformin, and buformin hydrochloride;
xxv. a cationic polymeric antimicrobial agent;
xxvi. a polymeric biguanide;
xxvii. an agent, selected from the group consisting of, abomycin, acetomycin, acetoxycycloheximide, acetylnanaomycin, an actinoplanes sp. Compound, actinopyrone, aflastatin, albacarcin, albacarcin, albofungin, albofungin, alisamycin, alpha-R,S-methoxycarbonylbenzylmonate, altromycin, amicetin, amycin, amycin demanoyl compound, amycine, amycomycin, anandimycin, anisomycin, anthramycin, anti-syphilis imune substance, anti-tuberculosis imune substance, antibiotic from Eschericia coli, antibiotics from Streptomyces refuineus, anticapsin, antimycin, aplasmomycin, aranorosin, aranorosinol, arugomycin, ascofuranone, ascomycin, ascosin, Aspergillus flavus antibiotic, asukamycin, aurantinin, an Aureolic acid antibiotic substance, aurodox, avilamycin, azidamfenicol, azidimycin, bacillaene, a Bacillus larvae antibiotic, bactobolin, benanomycin, benzanthrin, benzylmonate, bicozamycin, bravomicin, brodimoprim, butalactin, calcimycin, calvatic acid, candiplanecin, carumonam, carzinophilin, celesticetin, cepacin, cerulenin, cervinomycin, chartreusin, chloramphenicol, chloramphenicol palmitate, chloramphenicol succinate sodium, chlorflavonin, chlorobiocin, chlorocarcin, chromomycin, ciclopirox, ciclopirox olamine, citreamicin, cladosporin, clazamycin, clecarmycin, clindamycin, coliformin, collinomycin, copiamycin, corallopyronin, corynecandin, coumermycin, culpin, cuprimyxin, cyclamidomycin, cycloheximide, dactylomycin, danomycin, danubomycin, delaminomycin, demethoxyrapamycin, demethylscytophycin, dermadin, desdamethine, dexylosyl-benanomycin, pseudoaglycone, dihydromocimycin, dihydronancimycin, diumycin, dnacin, dorrigocin, dynemycin, dynemycin triacetate, ecteinascidin, efrotomycin, endomycin, ensanchomycin, equisetin, ericamycin, esperamicin, ethylmonate, everninomicin, feldamycin, flambamycin, flavensomycin, florfenicol, fluvomycin, fosfomycin, fosfonochlorin, fredericamycin, frenolicin, fumagillin, fumifungin, funginon, fusacandin, fusafungin, gelbecidine, glidobactin, grahamimycin, granaticin, griseofulvin, griseoviridin, grisonomycin, hayumicin, hayumicin, hazymicin, hedamycin, heneicomycin, heptelicid acid, holomycin, humidin, isohematinic acid, karnatakin, kazusamycin, kristenin, L-dihydrophenylalanine, a L-isoleucyl-L-2-amino-4-(4′-amino-2′,5′-cyclohexadienyl) derivative, lanomycin, leinamycin, leptomycin, libanomycin, lincomycin, lomofungin, lysolipin, magnesidin, manumycin, melanomycin, methoxycarbonylmethylmonate, methoxycarbonylethylmonate, methoxycarbonylphenylmonate, methyl pseudomonate, methylmonate, microcin, mitomalcin, mocimycin, moenomycin, monoacetyl cladosporin, monomethyl cladosporin, mupirocin, mupirocin calcium, mycobacidin, myriocin, myxopyronin, pseudoaglycone, nanaomycin, nancimycin, nargenicin, neocarcinostatin, neoenactin, neothramycin, nifurtoinol, nocardicin, nogalamycin, novobiocin, octylmonate, olivomycin, orthosomycin, oudemansin, oxirapentyn, oxoglaucine methiodide, pactacin, pactamycin, papulacandin, paulomycin, phaeoramularia fungicide, phenelfamycin, phenyl, cerulenin, phenylmonate, pholipomycin, pirlimycin, pleuromutilin, a polylactone derivative, polynitroxin, polyoxin, porfiromycin, pradimicin, prenomycin, Prop-2-enylmonate, protomycin, Pseudomonas antibiotic, pseudomonic acid, purpuromycin, pyrinodemin, pyrrolnitrin, pyrrolomycin, amino, chloro pentenedioic acid, rapamycin, rebeccamycin, resistomycin, reuterin, reveromycin, rhizocticin, roridin, rubiflavin, naphthyridinomycin, saframycin, saphenamycin, sarkomycin, sarkomycin, sclopularin, selenomycin, siccanin, spartanamicin, spectinomycin, spongistatin, stravidin, streptolydigin, streptomyces arenae antibiotic complex, streptonigrin, streptothricins, streptovitacin, streptozotocine, a strobilurin derivative, stub omycin, sulfamethoxazol-trimethoprim, sakamycin, tejeramycin, terpentecin, tetrocarcin, thermorubin, thermozymocidin, thiamphenicol, thioaurin, thiolutin, thiomarinol, thiomarinol, tirandamycin, tolytoxin, trichodermin, trienomycin, trimethoprim, trioxacarcin, tyrissamycin, umbrinomycin, unphenelfamycin, urauchimycin, usnic acid, uredolysin, variotin, vermisporin, verrucarin and analogs and salts and derivatives thereof.
xxviii. a naturally occurring antibiotic compound, selected from the group consisting of phenol, resorcinol, antibiotic aminoglycosides, anamycin, quinines, anthraquinones, antibiotic glycopeptides, azoles, macrolides, avilamycin, agropyrene, cnicin, aucubin antibioticsaponin fractions, berberine (isoquinoline alkaloid), arctiopicrin (sesquiterpene lactone), lupulone, humulone (bitter acids), allicin, hyperforin, echinacoside, coniosetin, tetramic acid, imanine, and novoimanine;
xxix. a plant oil or extracts which contain antibiotic agents;
xxx. an oil or extract of a plant selected from the group consisting of thyme, perilla, lavender, tea tree, terfezia claveryi, micromonospora, putterlickia verrucosa, putterlickia pyracantha putterlickia retrospinosa, maytenus ilicifolia, maytenus evonymoides, maytenus aquifolia, faenia interjecta, cordyceps sinensis, couchgrass, holy thistle, plantain, burdock, hops, echinacea, buchu, chaparral, myrrh, red clover, yellow dock, garlic, and St. John's wort; and esters and salts thereof.

107. The composition of claim 105, wherein the concentration range of the antibiotic agent is selected from the group of (i) between about 0.005% and about 0.5%; (ii) between about 0.5% and about 2%; (iii) between about 2% and about 5%; and (iv) between about 5% and about 12%.

108. The composition of claim 107, wherein the composition has an anti-inflammatory effect.

109. The composition of claim 93, wherein the solubility of the antibiotic agent in the aqueous phase of the emulsion is less than 1 parts of solvent required for 1 part of solute.

110. The composition of claim 93, wherein the antibiotic agent is dissolved in at least one phase of the emulsion.

111. The composition of claim 92, wherein the foamable carrier further contains at least one additional therapeutic agent selected from the group consisting of an a steroidal anti-inflammatory agent, an immunosuppressive agent, an immunomodulator, an immunoregulating agent, a hormonal agent, an antifungal agent, an antiviral agent, an antiparasitic agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, benzoyl chloride, calcium hypochlorite, magnesium hypochlorite, an anti-wrinkle agent, a radical scavenger, a metal, silver, a metal oxide, titanium dioxide, zinc oxide, zirconium oxide, iron oxide, silicone oxide, talc, carbon, an anti wrinkle agent, a skin whitening agent, a skin protective agent, a masking agent, an anti-wart agent, a refatting agent, a lubricating agent, and mixtures of any two or more thereof.

112. The composition of claim 92, wherein the concentration of the surface-active agent is between about 0.1% and about 5% by weight of the carrier, or is between about 0.2% to about 2% by weight of the carrier.

113. The composition of claim 92, wherein the surface-active agent consists essentially of a non-ionic surfactant, wherein the emulsion is
(i) an oil-in-water emulsion,
wherein the HLB range of the non-ionic surface-active agent is between about 9 and about 14; or
(ii) a water-in-oil emulsion,
wherein the HLB range of the non-ionic surface-active agent is between about 2 and about 9.

114. The composition of claim 91, wherein the surface-active agent comprises a mixture of at least one non-ionic surfactant and at least one ionic surfactant in a ratio in the range selected from the group consisting of about 100:1 to about 1:1, about 1:1 to 20:1, a ratio greater than about 8:1, and a ratio greater than about 16:1.

115. A composition comprising a foamable carrier and a propellant in a canister the carrier comprising:
i. an antibiotic agent; wherein the antibiotic agent is soluble in the composition or is substantially insoluble in water;
ii. an unsaturated fatty acid selected from the group consisting of an omega-3 fatty acid, an omega-6 fatty acid, and mixtures of both;
iii. about 0.1% to about 5% by weight of the carrier of a fatty alcohol;
iv. a fatty acid selected from the group consisting of fatty acids having 16 or more carbons in their carbon chain; fatty acids having at least one double bond;
branched fatty acids; fatty acids substituted with a hydroxyl group; hexadecanoic acid; stearic acid; arachidic acid; behenic acid; octacosanoic acid; 12-hydroxy stearic acid; and mixtures of any two or more thereof;
v. an emollient;
vi. about 0.1% to about 5% by weight of the carrier of a surface-active agent; and
vii. water
wherein the propellant is a liquefied or compressed gas propellant at a concentration of about 3% to about 25% by weight of the composition; and
wherein upon release a shear-sensitive foam is produced.

116. The composition of claim 115, further comprising a hydrophobic organic carrier at a concentration of about 2% to 50% by weight of the carrier;
wherein the emollient is in an amount selected from the group consisting of (i) about 2% to about 5%; (ii) about 5% to about 10%; and (iii) about 10% to about 20% by weight of the carrier; and
wherein the unsaturated fatty acid comprises docosahexaenoic acid (DHA).
